(19) United States
(12) Patent Application Publication
(10) Pub. No.: US 2024/0133854 A1
(43) Pub. Date: Apr. 25, 2024

(54) THERMAL FLUID SENSOR

(71) Applicant: Flusso Limited, Cambridge Cambridgeshire (GB)

(72) Inventors: Syed Zeeshan ALI, Cambridge Cambridgeshire (GB); Cerdin LEE, Trumpington Cambridgeshire (GB); Ethan GARDNER, Kineton Warwickshire (GB); Jonathan HARDIE, Cambridge Cambridgeshire (GB); Jon CALLAN, Cambridge Cambridgeshire (GB); Florin UDREA, Cambridge Cambridgeshire (GB); Daniel POPA, Cambridge Cambridgeshire (GB); Claudio FALCO, Cambridge Cambridgeshire (GB); Julian William GARDNER, Kineton Warwickshire (GB); Sean Dixon, Cambridge Cambridgeshire (GB)

(73) Assignee: Flusso Limited, Cambridge Cambridgeshire (GB)

(21) Appl. No.: 18/394,622

(22) Filed: Dec. 22, 2023

Related U.S. Application Data
(63) Continuation-in-part of application No. PCT/EP2022/067036, filed on Jun. 22, 2022.

(51) Int. Cl. G01N 33/00; G01M 3/04; G01N 1/44; G01N 27/04; G01N 27/22

(52) U.S. Cl. CPC G01N 33/0027; G01M 3/04; G01N 1/44; G01N 27/04; G01N 27/22

(57) ABSTRACT

A fluid sensor for sensing a concentration or composition of a fluid, the sensor comprising a first temperature sensing element located on or within a first dielectric membrane and a second temperature sensing element located on or within a second dielectric membrane. An output circuit is configured to measure a differential signal between the first temperature sensing element and the second temperature sensing element. The fluid sensor comprises a first region configured to be exposed to the fluid, and a second region configured to be isolated from the fluid, where the first dielectric membrane is located in the first region, such that in use, the first dielectric membrane is exposed to the fluid, and wherein the second dielectric membrane is located in the second region such that in use, the second dielectric membrane is isolated from the fluid.

### TECHNICAL FIELD

The present disclosure relates to a micro-machined sensor, particularly but not exclusively, the disclosure relates to a fluid sensor for sensing concentration of a fluid or concentration of components of a fluid based on thermal conductivity of the fluid.

### BACKGROUND

There is an increasing demand for gas sensors to monitor pollutants in our environment. Gas sensors can be based on many different principles and technologies. One such principle is using thermal conductivity to determine the composition of gases.

For example, in G. De Graaf and R. F. Wolffenbuttel, “Surface-micromachined thermal conductivity detectors for gas sensing.” 2012 IEEE International Instrumentation and Measurement Technology Conference Proceedings, pp. 1861-1864, a thermal conductivity gas sensor based on silicon technology is described.

Mandavifar et.al. in “Simulation and Fabrication of an Ultra-Low Power miniature Microbridge Thermal Conductivity Gas Sensor,” Journal of the Electrochemical Society, 161 B55, describe a device comprising a suspended thin polysilicon resistor that acts as a heater and a temperature sensor as part of a thermal conductivity sensor. The change in resistance of the polysilicon with temperature allows its use as a temperature sensor.

U.S. Pat. Nos. 10,598,621, 8,667,839B2, and U.S. 63/572,279B1, US86889608 and U.S. Pat. No. 10,408,802B2 describe further sensors. Kommandur et. al., “A microbridge heater for low power gas sensing based on the 3-omega technique,” Sensors and Actuators A 233 (2015) 231-238, also describes a thermal conductivity sensor.

### SUMMARY

Presently available sensors have, among others, the following disadvantages:
high power dissipation, low sensitivity and slow dynamic response of the sensor;
mechanical fragility and vibration sensitivity;
reduced mechanical robustness of sensor supporting structures;
complex fabrication processes;
manufacturing processes that are not fully CMOS compatible; and
manufacturing processes that are expensive.

The devices of the present disclosure are advantageous over the state-of-the-art devices for the following reasons:
the sensor is able to determine composition of a fluid and concentration of different components within the fluid, in a zero flow environment;
thermal isolation of the heated element which reduces power dissipation, increases sensitivity and provides a fast, dynamic response of the sensor;
reduced mechanical fragility and vibration sensitivity of the membrane structure compared to a beam structure;
a suitable dielectric material used for the dielectric membrane improves mechanical robustness of the membrane;
a suitable dielectric material used for the dielectric membrane reduces power dissipation, increases sensitivity and provides a fast, dynamic response of the sensor;
discontinuities within the membrane mitigate power dissipation, sensitivity and dynamic response issues; and
the devices are fully CMOS compatible and therefore can be manufactured using fully CMOS compatible processes.

The presently disclosed fluid sensor is able to measure the composition of the fluid based on the different thermal conductivity of each of the components of the fluid.

Aspects and preferred features are set out in the accompanying claims.

According to an aspect of the disclosure, there is provided a fluid sensor for sensing a concentration or composition of a fluid, the sensor comprising:
a semiconductor substrate comprising a first substrate portion and a second substrate portion, the first substrate portion comprising a first cavity and the second substrate portion comprising a second cavity;
a dielectric region located on the semiconductor substrate, wherein the dielectric region comprises a first dielectric membrane provided on the first cavity and a second dielectric membrane provided on the second cavity;
a first temperature sensing element located on or within the first dielectric membrane and a second temperature sensing element located on or within the second dielectric membrane;
an output circuit configured to measure a differential signal between the first temperature sensing element and the second temperature sensing element,
   wherein, the fluid sensor comprises a first region configured to be exposed to the fluid, and a second region configured to be isolated from the fluid,
   wherein the first dielectric membrane is located in the first region, such that in use, the first dielectric membrane is exposed to the fluid, and
   wherein the second dielectric membrane is located in the second region such that in use, the second dielectric membrane is isolated from the fluid.

According to a further aspect of the disclosure, there is provided a fluid sensor for sensing a concentration or composition of a fluid, the sensor comprising:
a semiconductor substrate comprising a first substrate portion and a second substrate portion, the first substrate portion comprising a first cavity and the second substrate portion comprising a second cavity, wherein the first cavity and the second cavity are separate cavities;
a dielectric region located on the semiconductor substrate, wherein the dielectric region comprises a first dielectric membrane provided on the first cavity and a second dielectric membrane provided on the second cavity;
   wherein, the fluid sensor comprises a first region configured to be exposed to the fluid, and a second region configured to be isolated from the fluid,
   wherein the first dielectric membrane is located in the first region, such that in use, the first dielectric membrane is exposed to the fluid, and
   wherein the second dielectric membrane is located in the second region such that in use, the second dielectric membrane is isolated from the fluid.

According to a further aspect of the disclosure, there is provided a fluid sensor for sensing a concentration or composition of a fluid, the sensor comprising:
a substrate comprising at least one semiconductor layer, wherein the substrate comprises a first substrate portion and a second substrate portion, the first substrate portion comprising a first cavity and the second substrate portion comprising a second cavity;
a dielectric region located on the semiconductor substrate, wherein the dielectric region comprises a first dielectric membrane provided on the first cavity and a second dielectric membrane provided on the second cavity;
a first temperature sensing element located on or within the first dielectric membrane and a second temperature sensing element located on or within the second dielectric membrane;
an output circuit configured to measure a differential signal between the first temperature sensing element and the second temperature sensing element,
   wherein, the fluid sensor comprises a first region configured to be exposed to the fluid, and a second region configured to be isolated from the fluid,
   wherein the first dielectric membrane is located in the first region, such that in use, the first dielectric membrane is exposed to the fluid, and
   wherein the second dielectric membrane is located in the second region such that in use, the second dielectric membrane is isolated from the fluid.

The first cavity may be a first etched portion and the second cavity may be a second etched portion.

The fluid sensor may comprise a first set of temperature sensing elements each located on the first membrane, wherein the first set of temperature elements comprises the first temperature sensing element and a third temperature sensing element; and a second set of temperature sensing elements each located on the second membrane, wherein the second set of temperature elements comprises the second temperature sensing element and a fourth temperature sensing element.

In other words, the first dielectric membrane may be exposed to a surrounding fluid while the second dielectric membrane may be sealed so as to be isolated from the surrounding fluid. The output circuit may determine the concentration or composition of the fluid based on a thermal conductivity of the fluid.

The first temperature sensing element and/or the second temperature sensing element may be configured to operate as heating elements.

One or both of the temperature sensing elements within the first and second dielectric membranes may be configured to operate as heating elements.

Additionally or alternatively, the first and second dielectric membranes may each comprise an additional heating element.

In other words, one of the temperature sensing elements in each of the dielectric membrane may also configured to operate as a heating element. Alternatively, each dielectric membrane may have a separate heating element.

The output circuit may be configured to measure a differential signal between the first set of temperature sensing elements and the second set of temperature sensing elements. In other words, the output circuit may be configured to measure a differential signal between the two temperature sensing elements of the first dielectric membrane and the two temperature sensing elements of the second dielectric membrane.

The output circuit may comprise one or more of: a constant current source, a differential amplifier, an instrumentation amplifier, an analogue to digital convertor and a micro-controller.

The output circuit may comprise a Wheatstone bridge. Differential signals can be obtained by using a combination of current sources and differential amplifiers, bridge type circuits or other types of subtraction circuits or instrumentation amplifiers.

The Wheatstone bridge may be arranged to be balanced by a change of a bias of one or more of a heating element, or temperature sensing element operating as a heating element, on the first or second dielectric membranes.

The two temperature sensing elements from the first dielectric membrane or from the second dielectric membrane may operate as respective first and second legs of the Wheatstone bridge.

The sensor may further comprise an ambient temperature sensor on the semiconductor substrate.

An additional or further temperature sensor (referred to as an ambient temperature sensor) may be placed outside the dielectric membrane as a reference temperature sensing element to measure the ambient temperature or the temperature of the fluid, and the signal from the further temperature sensor may be used for temperature compensation for a more accurate calculation of the concentration of one or more specific components of the fluid. While the use of two membranes and a Wheatstone bridge helps cancel out many common mode effects such as temperature, changes in ambient temperature can still cause changes in device sensitivity. A separate measurement of ambient temperature thus allows more accurate determination of the fluid composition.

The reference temperature sensing element could be integrated on-chip as an extra resistive temperature detector, a diode or a transistor. An ambient temperature sensor could also be provided as part of the ASIC as a VPTAT or IPTAT sensor based on bandgap reference.

The temperature compensation can be done by using both the temperature reading from the additional temperature sensing element and the differential reading between the temperature sensing elements within the first and second dielectric membranes. This can be implemented by either a formula (within an algorithm) to adjust the final reading, or using a look up table and interpolation to determine the final reading.

The ambient temperature sensor may operate as a respective third or fourth leg of the Wheatstone bridge.

The two temperature sensing elements from the other of the first or second dielectric membrane may operate as respective third and fourth legs of the Wheatstone bridge. In other words, the first set of temperature sensing elements may operate as the first and second legs of the Wheatstone bridge and the second set of temperature sensing elements may operate as the third and fourth legs of the Wheatstone bridge. As will be described below, filling all four legs of a Wheatstone bridge with said temperature sensors allows for a greatly improved device sensitivity.

Implementations are explained in more detail as follows. The sensor may be provided with first and second identical dielectric membranes, identical heating elements on the first and second dielectric membranes, and two resistive temperature sensing elements each on the first and second dielectric membranes that are identical in terms of size, shape and materials. For most electrically conductive materials the electrical resistance changes with temperature, so the change in resistance of the resistive temperature sensing elements corresponds to the change in temperature of the membrane. Preferably, the temperature sensing elements may be made with a material having a high Temperature Coefficient of Resistance (TCR) so as to have a high sensitivity.

In operation, the heating elements of the first and second dielectric membrane may be biased to heat up the membranes to a high temperature. The bias to the heater can be a constant current, constant voltage or constant power bias. Because the first dielectric membrane is exposed to the surrounding fluid, change in thermal conductivity of the surrounding fluid (due to change in fluid composition) will cause a change in the heat loss from the membrane, and hence a temperature change on the first dielectric membrane. Because the second dielectric membrane is isolated from the surrounding fluid, it is not affected by the change in fluid composition. Hence, the difference in temperature between the first and second membrane will be affected by change in fluid composition, and will be detected by the readout circuit comprising the four temperature sensing elements.

Advantageously, the resistive temperature sensing elements may be connected as part of a readout or output circuit, with the four temperature sensing elements operating as legs of a Wheatstone bridge. The two temperature sensing elements from the first dielectric membrane are envisaged to be on opposite sides to each other within the Wheatstone bridge, and similarly the two temperature sensing elements from the second dielectric membrane will be on opposite sides to each other within the Wheatstone bridge. When the temperatures on both the membranes are the same, the Wheatstone bridge will output zero volts. But when the temperature on one of the membranes changes, then the Wheatstone bridge will be unbalanced and will give an output voltage. Further circuitry such as an instrumentation amplifier and analogue to digital convertor may also be present.

Compared to providing only one temperature sensing element on each membrane, providing two temperature sensing elements on each dielectric membrane, allows all four branches or legs of the Wheatstone bridge to be filled with said temperature sensing elements, thereby doubling the sensitivity of the device compared to a scenario where only two legs of a single Wheatstone bridge are filled with temperature sensing elements (the other legs being filled by, for example, known value resistors).

Another way to operate the device is using a feedback loop. The four temperature sensing elements may be part of a Wheatstone bridge whose output is normally zero. In presence of the target gas the heating element on the first membrane will have lower or higher thermal losses, and hence the temperature of the first membrane will change causing the Wheatstone bridge to be unbalanced and give an output value based on the change in temperature. A feedback control circuit can then be used to change the bias on the heating element on the first membrane until the bridge is balanced again and gives a zero output. The bias on the heating element is then used as the value to determine the composition of the surrounding fluid.

While ideally the heating elements should be identical, and the temperature sensing elements should be identical, in practice there may be small differences due to fabrication tolerances. This can be accounted for by calibration of the device in known conditions before use. Based on the calibration, the device may then store the output value in the known conditions and use that value during measurements to offset against the measured value. Alternatively during calibration the heating elements on the first and/or membrane maybe biased slightly differently until the output is zero. The required bias value to get zero output in the calibration condition may then be stored, and the same bias used on the first and/or second membrane during measurement.

The temperature sensing elements may be located in a same layer of the dielectric region as the heating element and the temperature sensing elements may laterally surround the heating element.

Alternatively, the temperature sensing elements may be located below or above the heating element. The temperature sensing elements may be located directly above or below the heating element, so that the temperature sensing elements are not laterally spaced from the heating element.

Having the temperature sensing elements below or above the heating element has the advantage that the temperature of the temperature sensing elements is substantially the same of that of the heater. This increases the differential signal between the temperature sensing elements of the first and second membrane in the presences of target fluid, therefore improving sensitivity of the sensor. If the temperature sensing elements are laterally spaced from the heating element then it is preferred that they be as close together as possible to improve sensitivity.

As described above, one of the resistive temperature sensing elements in each of the dielectric membranes may also act as the heating element. In this case, electronic switches can be used to temporarily disconnect the heating elements from the applied bias and be used to measure the bridge output. The measurement time in this case should be much shorter than the thermal time constant of the membrane, so the turn off time doesn't affect the temperature of the membrane. The advantage of this embodiment is simplicity and reduced number of additional elements on the membrane. The larger the number of elements on the dielectric membrane, the higher the probability of impaired reliability or malfunction of the sensor. The reduced number of elements can also result in reduced power consumption.

Alternatively, both of the temperature sensing elements in each of the dielectric membranes may also act as heating elements. In this case, the temperature sensing elements may also be part of a Wheatstone bridge, and the bias applied to the Wheatstone bridge may also heats up the membranes. The circuitry in this embodiment can be much simpler than the previous embodiments.

The Wheatstone bridge may be formed on a same chip as the fluid sensor. The sensor chip may comprise circuitry or connections. For example, the temperature sensors may be connected up in a Wheatstone bridge on the sensor chip. Drive circuitry, signal processing and read-out circuitry may be monolithically integrated or provided on a separate ASIC chip.

The Wheatstone bridge may comprise one or two active fluid sensors and one or two temperature sensors and/or passive components or reference cells or reference sensors which are not exposed to the fluid or are encapsulated in a fluid with a known concentration and pressure.

The first set of temperature sensing elements and the second set of temperature sensing elements may be directly connected to each other on or over the dielectric layer or semiconductor substrate.

The fluid sensor may further comprise a first fluid sensitive layer located on or over the first dielectric membrane; a second fluid sensitive layer located on or over the second dielectric membrane; a first electrode in contact with the first fluid sensitive layer; and a second electrode in contact with the second fluid sensitive layer.

The fluid sensor may further comprise a control unit configured to receive a resistive or capacitive reading from the first electrode and/or the second electrode. The control unit may be configured to calibrate the sensor using the received resistive or capacitive reading.

The fluid sensor may comprise a first set of electrodes in contact with the first fluid sensitive layer and a second set of electrodes in contact with the second fluid sensitive layer. The first set of electrodes may comprise the first electrode and a third electrode separated from each other by at least a portion of the first fluid sensitive layer. The second set of electrodes may comprise the second electrode and a fourth electrode separated from each other by at least a portion of the second fluid sensitive layer.

A resistance or capacitance of the first fluid sensitive layer and the second fluid sensitive layer may be dependent on humidity.

The semiconductor substrate may comprise separate first and second semiconductor substrate sections.

The semiconductor substrate may comprise integral first and second substrate sections. The first dielectric membrane and the second dielectric membrane may comprise integral sections of a dielectric layer.

The first semiconductor substrate section may be located in the first region (or exposed region) and the second semiconductor substrate section may be located in the second region (or sealed region).

The fluid sensor may comprise a controller configured to:
drive one or more of the heating elements in an AC mode to modulate the temperature of the heating elements to vary the differential signal;
monitor the differential signal at the modulation frequency using a lock-in amplifier and/or based on a Fourier transform-based technique; and
selectively differentiate between different fluid components and/or determine the concentration of the different fluid components based on the differential signal.

The fluid sensor may comprise two DC current sources configured to independently generate DC currents with alternating polarities; and a controller configured to:
drive one or more of the heating elements or a sensing element adjacent to the heating element in using DC pulses from the two DC current sources; and
monitor the differential signal based on a two point or a three point DC reversal-based technique; and
selectively differentiate between different fluid components and/or determine the concentration of the different fluid components based on a differential signal.

The fluid sensor may comprise one or more current sources with alternating polarities, and a controller configured to:
drive one or more of the heating elements using a current from the one or more current sources; and
monitor the differential signal based on a two point or a three point DC reversal-based technique; and
selectively differentiate between different fluid components and/or determine the concentration of the different fluid components based on a differential signal.

One or more of the heating elements may be driven by a single current source. The fluid sensor may comprise one or more switches configured to change the direction of a current in terminals of one or more of the heating elements or one or more of the temperature sensing elements.

The fluid sensor may comprise a control unit configured to drive one or more of the heating elements in an AC bias or a pulse bias, and further configured to determine the concentration and type of gas or gases present based on the frequency content of a resulting signal.

A first ambient temperature sensor may be close to but outside of the first dielectric membrane and a second ambient temperature sensor may be close to but outside of the second dielectric membrane. The first ambient temperature sensor may be provided in addition to the two temperature sensing elements provided on or within the first dielectric membrane, while the second ambient temperature sensor may be provided in addition to the two temperature sensing elements provided on or within the second dielectric membrane.

The fluid sensor may further comprise a first ambient temperature sensor close or adjacent to the first dielectric membrane and a second ambient temperature sensor close or adjacent to the second dielectric membrane. The fluid sensor may be configured to determine a measurement of the fluid concentration or composition based on at least one of:
(i) a signal from the first ambient temperature sensor;
(ii) a signal from the second ambient temperature sensor; and/or
(iii) a differential signal between the first ambient temperature sensor and the second ambient temperature sensor.

The fluid sensor may further comprise a first ambient temperature sensor and a second ambient temperature sensor. The first ambient temperature sensor may be located in the first region, and the second ambient temperature sensor may be located within the second region. The fluid sensor may be configured to determine a measurement of the fluid concentration or composition based on at least one of:
(i) a signal from the first ambient temperature sensor;
(ii) a signal from the second ambient temperature sensor; and/or
(iii) a differential signal between the first ambient temperature sensor and the second ambient temperature sensor.

A direct or a differential measurement of the first and second ambient temperature sensors may be made, for example by readout circuitry of the system, and the values may be used along with the differential reading between the temperature sensing elements provided on or within the first and second dielectric regions, to determine a more accurate value of the fluid composition and/or a concentration of a target gas. Advantageously, this method may account for differences that occur in the ambient temperature between the first and second dielectric membranes. In implementations in which the first and second dielectric membranes are close to each other, the difference in the ambient temperature at or around the first dielectric membrane and the ambient temperature at or around the second dielectric membrane will generally remain relatively small. However, in some implementations even a small difference in the ambient temperature at these locations can result in or contribute to a significant error in measurement results, such as a gas concentration measurement. As a result, by measuring the ambient temperatures at the first and second dielectric membranes, or otherwise determining a difference in the ambient temperature at the first dielectric membrane and the ambient temperature at the second dielectric membrane, the accuracy of the measurement result may be improved.

In another arrangement, there may be provided two first temperature sensors close to but outside of the first dielectric membrane, and two second temperature sensors close to but outside of the second dielectric membrane. All four of the first and second temperature sensors located outside of the dielectric membranes may be connected together, e.g. in a Wheatstone bridge, to determine a differential measurement between the four first and second sensors. This arrangement may also provide an analog signal that can be subtracted from the main sensor signal using e.g. a differential amplifier, to further improve the sensor response. As above, the two first/second temperature sensors may be provided in addition to the two temperature sensing elements provided on or within the first/second dielectric membrane.

The fluid sensor may comprise a measurement system configured to:
store one or more variables;
receive a reading from one or more of the temperature sensing elements;
determine the fluid composition based on the reading and the one or more variables; and
update the one or more variables based on the reading and/or the determined fluid composition.

The first region may comprise an exposed region that is exposed to the fluid to be sensed, and the second region may comprise a sealed region. The sealed region may be substantially or completely sealed. The sealed region may be isolated from the fluid to be sensed. The second region may comprise a sealed region comprising a fluid of a known composition.

The sealed region may be sealed containing one or more of: air, dry air, synthetic air, an inert gas such as nitrogen or argon, a vacuum, or a partial vacuum or any other fluid mixture of known quantity. The sealed region can be a fully hermetic seal, or a partially hermetic seal. A fully hermetic seal may fully isolate the fluid from the external environment, such that the fluid is not affected by the outside atmosphere. A partially hermetic seal may generally allow a small or slow leak or transfer of the internal atmosphere and external atmospheres, and as such the fluid may be affected by the conditions outside of the sealed region when measured over a long period of time (for example, days, weeks or months).

The sealed region may contain a gas with a known composition. For example, if the CO₂ concentration in air is to be detected by the fluid sensor of the present disclosure, to the sealed region may comprise a known concentration of CO₂. This concentration could be any known level from its typical concentration in air 400 ppm to any level up to 100% CO₂.

The second dielectric membrane can be isolated from the surrounding fluid by encapsulating it with a known fluid. The encapsulation can be done on wafer/chip level, or on a package level. The term package is used herein to refer to an assembly or module comprising the fluid sensor and one or more other components mounted to, on or with the fluid sensor, and/or coupled thereto.

In one example, there may be a cap with sidewalls and lid attached above the second dielectric membrane, and also a base below the substrate closing off the second cavity portion of the substrate. In this way the second dielectric membrane is isolated from the environment, and will only be in contact with the fluid that it is encapsulated with. The cap and base could be made of a semiconducting material such as silicon. They can also be made of glass, metal or plastic. The cap could also comprise sidewalls made of silicon and lid made of dielectric materials such as silicon dioxide and silicon nitride. Such an encapsulation can be done at wafer level. The encapsulation may be done by any suitable wafer bonding methods, such as fusion bonding, anodic bonding, glass-frit bonding, adhesive bonding, and/or eutectic bonding.

In another example the encapsulation is done on a package level. In this case, the package comprises a base, and a cap such that the package comprises two regions—one region which is sealed, and one which is exposed to the surrounding fluid. The package cap in the exposed region may have one or more holes in it to allow exposure to the surrounding fluid. There may also be a filter in the holes to protect against dust or moisture. A package wall separates these regions. The sensor is packaged such that part of the semiconductor substrate is in the sealed region, and part of the semiconductor substrate is in the exposed region, with first dielectric membrane within the exposed region, and the second dielectric membrane within the sealed region.

In another example of encapsulation done on package level, the there are two semiconductor substrates, the first semiconductor substrate having a first etched portion and first dielectric membrane, and a second semiconductor substrate having a second etched portion and the second dielectric membrane. The first semiconductor substrate is in the exposed portion of the package, and the second semiconductor substrate is in the sealed region of the package.

The semiconductor substrate may further comprise a third substrate portion comprising third etched region or cavity. The dielectric region may further comprise a third dielectric region provided on the third etched portion. The fluid sensor may comprise a third temperature sensing element located on or within the third dielectric membrane. The third dielectric membrane may be located in a third region configured to be isolated from the fluid, and the third region may comprise a sealed region comprising a vacuum.

The first and/or second and/or third dielectric membranes may have the same size and shape, and the temperature sensing elements from the first and/or second and/or third dielectric membranes may have the same size and shape.

The temperature sensing elements may comprise one or more of: resistors, diodes, transistors, thermopiles, or a combination thereof.

The heating elements may comprise resistors or transistors.

The sensor may comprise an application specific integrated circuit (ASIC).

According to a further aspect of the disclosure, there is provided a sensor assembly comprising the fluid sensor as described above and an application specific integrated circuit (ASIC) coupled to the sensor.

The control circuitry can be located on the same chip as the sensor (monolithically integrated), or can have an application specific integrated circuit (ASIC) coupled to the sensor. The ASIC can be on a separate chip, but within the same package, as a hybrid, co-packaged or using system in package (SIP) solutions. Alternatively, the ASIC could be placed outside the package, on a PCB (Printed Circuit Board) or within the same case/box.

The ASIC may be located underneath the sensor, for example using a die stack technique. Alternatively, the ASIC may be located side by side with the sensor or elsewhere. The ASIC may be connected to the sensor using wire bonding and pads, or using through-silicon-vias (TSV) extending through the semiconductor substrate. Alternatively, the sensor and the ASIC can be located on the surface of a common PCB or embedded in a PCB.

An ASIC may be provided within the same system or the same package or on-chip to provide electronic circuitry to drive, read-out signals and process signals from the sensor. The ASIC may be placed in a stack die configuration under the sensor and the sensor and ASIC are placed within a manifold or an open package, to allow contact to the fluid.

The ASIC may operate as a base of the sensor and may comprise one or more through-silicon vias for making electrical connections.

According to a further aspect of the disclosure, there is provided a sensor assembly comprising a sensor housing; and a fluid sensor as described above located within the flow sensor housing.

The sensor may be arranged in a flip-chip configuration.

According to a further aspect of the disclosure, there is provided a sensor assembly comprising the fluid sensor as described above, wherein the fluid sensor may be packaged on a printed circuit board in a flip-chip configuration.

The device may be packaged in a metal TO type package, in a ceramic, metal or plastic SMD (surface mount device) package. The device may also be packaged directly on a PCB, or with a flip-chip method. The device may also be embedded in a substrate, such as a customised version of one of the previously mentioned package, a rigid PCB, a semi-rigid PCB, flexible PCB, or any other substrate, in order to have the device surface flush with the substrate surface. The package can also be a chip or wafer level package, formed for example by wafer-bonding.

In particular, the package maybe designed such that there is a surface very close to the membrane, for example in a flip-chip scenario, such that the surface is less than 50 um from the membrane. This increases the power loss through the fluid and improves the sensitivity of the sensor.

The heating element may be a resistive heating element, made of a CMOS compatible material such as aluminium, tungsten, copper, titanium, polysilicon or single crystal silicon. It can also be made from other materials such as platinum or gold, or other semiconductor materials such as silicon carbide, gallium nitride, aluminium gallium nitride or gallium arsenide. Alternately the heating element could be a diode or a transistor.

The temperature sensing elements may be resistive temperature sensing elements and may comprise a resistor made of metal (such as tungsten, aluminium, copper, platinum, gold, titanium) or semiconductor material (such as silicon, polysilicon, silicon carbide, gallium nitride, aluminium gallium nitride, or gallium arsenide or a two dimensional electron gas).

The temperature sensing elements may preferably be identical in size, shape and resistance. Alternatively, they may be different within the first membrane, but be identical to the corresponding temperature sensing elements within the second membrane. In such a configuration it is easy to get a zero differential signal in the case that the target gas is not present.

Alternatively they may be of different size, shape and/or resistance. In this case the differential signals may not be zero when the target gas is not present, and either there is always an offset in the signal, or calibration maybe required.

For increased sensitivity and stability, such resistive temperature detectors may have a high, reproducible and stable TCR (Temperature Coefficient of Resistance). Secondly, it is preferable that such resistive temperature detectors are linear in temperature (i.e. their resistance varies linearly with the temperature).

However in other embodiments, the temperature sensing elements may not be resistive but can be diodes, or transistors. One type of temperature sensing element may be used, or a combination of different types of sensing elements may be used. For example, both the membrane may have one resistive temperature sensor each, and one diode temperature sensor each. When used as a combination such as this, the positions on the output circuit (such as a Wheatstone bridge) can also be different depending on the types of temperature sensors used so as to achieve the maximum sensitivity. In such a combination, one of the temperature sensors can also be a thermopile.

In case of diodes or transistors, the bridge circuit would comprise diodes or transistors instead of resistors. Resistors can be simpler to fabricate, while diodes & transistors can have the advantage of better process tolerances as compared to resistors.

The fluid sensor may comprise a semiconductor substrate made of a semiconductor material such as silicon, silicon carbide or Gallium Nitride, and comprising an etched portion. The fluid sensor may also comprise a dielectric region comprising of oxides and/or nitrides such as silicon dioxide and silicon nitride, where the portion of the dielectric region adjacent to the etched portion is referred to as a dielectric membrane. The dielectric membrane may have embedded structures made of semiconductor material or metal structures.

The semiconductor substrate may be any semiconductor such as silicon, silicon on insulator (SOI), silicon carbide, gallium nitride or diamond. In particular, the use of silicon is advantageous, as it guarantees sensor manufacturability in high volume, low cost and high reproducibility. The use of a silicon substrate could also enable on-chip circuitry for sensor performance enhancement and system integration facilitation. Such on-chip circuitry could be implemented by using analogue or digital or mixed-signal blocks placed outside the dielectric membrane.

The dielectric membrane or multiple dielectric membranes may be formed by back-etching using Deep Reactive Ion Etching (DRIE) of the substrate, which results in vertical sidewalls and thus enabling a reduction in sensor size and costs. However, the back-etching can also be done by using anisotropic etching such as KOH (Potassium Hydroxide) or TMAH (TetraMethyl Ammonium Hydroxide) which results in sloping sidewalls. The dielectric layers within the membrane which could be formed by oxidation or oxide deposition could be used as an etch stop during the DRIE or wet etching processes. The membrane can also be formed by a front-side etch (using most commonly wet etch techniques) or a combination of a front-side and back-side etch to result in a suspended membrane structure, supported only by two or more beams. The membrane may be circular, rectangular, or rectangular shaped with rounded corners to reduce the stresses in the corners, but other shapes are possible as well.

In a further disclosed aspect, instead of the etched portions of the semiconductor substrate or in addition to them, the sensor may comprise etched portions or cavities in layers located above the semiconductor substrate. In this case, an additional layer may be provided between the semiconductor substrate and the dielectric layer. Part of such additional layer may be removed to form a first cavity portion under the dielectric layer to define the first dielectric membrane, and a second cavity portion under the dielectric layer to define the second dielectric membrane.

The additional layer may comprise a single layer or a plurality of layers, comprising the same or different materials. Such material could be an insulating material (e.g. oxide, nitride based) or polycrystalline or monocrystalline semiconductor layer (e.g. polysilicon, silicon). The cavity portions may extend from a bottom surface to a top surface of the additional layer, over the entire thickness of the additional layer. Alternatively such cavity portions may not extend over the entire thickness. Optionally, and in addition to the cavity portions in the additional layer, there may be provided also an etched portion within the semiconductor substrate, for example, below the cavity portion(s) in the additional layer.

Preferably, the semiconductor substrate may be silicon and the dielectric membrane may be formed mainly of oxide and nitride materials, or oxinitride (a pre-formed combination of oxide and nitride) and where the heater element may be made of a metal such as tungsten, titanium, copper, aluminium, gold, platinum or a combination of those or a semiconductor such as highly doped n type or p type silicon or polysilicon. The heater may have a shape of a meander, spiral or a hotwire.

The dielectric region may comprise a dielectric layer or a plurality of layers including one or more dielectric layers. The dielectric region may comprise layers of more than one material, such as silicon dioxide, silicon nitride, or aluminium oxide. The heating element may be fully embedded or partially embedded within the dielectric membrane.

The membrane may also comprise one or more layers of spin on glass, and a passivation layer over the one or more dielectric layers. The employment of materials with low thermal conductivity (e.g. dielectrics) enables a significant reduction in power dissipation as well as an increase in the temperature gradients within the membrane with direct benefits in terms of sensor performance (e.g. sensitivity, frequency response, range, etc.). Temperature sensing elements or heaters made of materials such as monocrystalline or polycrystalline semiconductors or metals could be suspended or embedded in the dielectric membrane.

The dielectric membrane may also have other structures made of metal or other conductive or other materials with higher mechanical strength. These structures can be embedded within the membrane, or may be above or below the membrane, to engineer the thermo-mechanical properties (e.g. stiffness, temperature profile distribution, etc.) of the membrane and/or the fluid dynamic interaction between the fluid and the membrane. More generally, these structures can be also outside the membrane and/or bridging between inside and outside the membrane.

Generally speaking, a dielectric membrane region may be located immediately adjacent or above (or below if a flip-chip technology is used) to the etched portion of the substrate.

The dielectric membrane region corresponds to the area of the dielectric region directly above or below the etched cavity portion of the substrate. Each dielectric membrane region may be over a single etched portion of the semiconductor substrate. The membrane maybe a “closed membrane”, supported by the substrate along its entire perimeter, or can be a bridge type structure—supported by a number of dielectric beams. The membrane can be any shape—circular, square, rectangle, or square/rectangular with rounded corners.

The fluid sensor may be configured to sense or measure a fluid (this may be a gas but could also be a liquid), and the gas may be made of air and the components of interest could be any of CO₂, methane or hydrogen or other gases in dry air or humid air. The component of interest can be any fluid that has a different thermal conductivity than that of air.

The disclosed sensor could be applicable to a variety of gases and liquids, but we make specific reference to Carbon dioxide (CO₂), methane and hydrogen as these specific gases have thermal conductivity properties which are significantly different from those of air.

A control and measurement unit/circuitry that drives the heater in constant current, constant voltage or constant power mode may be provided. The driving could be preferably in pulse mode, but continuous mode or AC mode are possible.

The circuitry may be located on a same chip as the fluid sensor. Analogue/digital circuitry may be integrated on-chip. Circuitry may comprise IPTAT, VPTAT, amplifiers, analogue to digital converters, memories, RF communication circuits, timing blocks, filters or any other mean to drive the heating element, read out from the temperature sensing elements or electronically manipulate the sensor signals. For example, it is demonstrated that a heating element driven in constant temperature mode results in enhanced performance and having on-chip means to implement this driving method would result in a significant advancement of the state-of-the-art flow sensors. The driving method known a 3ω may be implemented via on-chip means, or any other driving method, such as constant temperature difference and time of flight, needed to achieve specific performance (e.g. power dissipation, sensitivity, dynamic response, range, fluid property detection, etc.). In absence of on-chip circuitry, this disclosure also covers the off-chip implementation of such circuital blocks when applied to a fluid sensor. Such off-chip implementation may be done in an ASIC or by discrete components, or a mix of the two.

The circuitry may include one or more alternating current (AC) sources and/or lock-in amplifier measurements to reduce noise. The one or more AC sources may be used in conjunction with fast Fourier transform (FFT)-based techniques. In some implementations, the use of lock-in amplifiers facilitate the measurements of very small AC signals, for example AC signals of a few nanovolts or less. Accurate measurements of the AC signals can be made even when noise sources are higher than the signal of interest. Because these techniques only measure AC signals at or near the test frequency, the effects of noise, including thermoelectric voltages (both DC and AC), may be reduced. The driving circuit may be implemented though any suitable means, such as via on-chip means, provided externally in an ASIC, a Field-programmable gate array (FPGA), micro-controller/micro-processor forms, by using discrete components, or any combination of the above means.

The circuitry may comprise reversible direct current (DC) sources. The reversible DC sources may be used in conjunction with techniques based on voltage measurements with currents of opposite polarity to cancel out thermoelectric noise (i.e., electromotive forces (EMFs)) that is generated when different parts of a circuit are at different temperatures, and/or when conductors made of dissimilar materials are joined together. Thermoelectric noise such as EMFs may be reduced or, in implementations, cancelled using consecutive voltage measurements made at alternating test current polarities.

The fluid sensor may be operated using a two voltage measurement method or a three voltage measurement method (also referred to as a ‘delta technique’), wherein two or three voltages are applied to any heating element(s) and/or the temperature sensing element(s). The two voltage measurement technique may reduce or cancel a thermoelectric voltage offset term from the measurement results. Similarly, the three voltage measurement method may reduce or cancel a thermoelectric voltage offset, and may additionally remove the thermoelectric voltage change (drifting) term from the measurement results, thus greatly improving the measurement noise immunity (e.g. the signal to noise ratio) when compared to many other techniques. The driving circuit may be implemented within the same chip (i.e. monolithic integration) or may be provided externally (such as in an off-chip implementation). The driving and reading circuits may be implemented with any suitable means, such as an ASIC, FPGA, by using discrete components, or any combination of the above. Output signals may be computed using any suitable processor and/or controller, such as a micro-controller or micro-processor.

Generally, this technique makes use of currents of equal (or approximately equal) magnitude and opposite polarities.

Opposite polarity currents can be used to more accurately measure the resistance and/or any change in the resistance of the heating element(s) (such as a heater) itself or any temperature sensing element(s) (e.g. a resistive temperature detector or thermal sensor) adjacent or otherwise close to the heater.

In some implementations, the opposite polarity currents are provided by opposite polarity current sources. One or more of the opposite polarity current sources may be e.g. precision current sources.

Additionally or alternatively, the opposite polarity currents may be provided by the same current source. This may be achieved by swapping the terminals of the (two-terminal) element to be measured. This element to be measured may be a heating element, such as a heating element used as a thermal sensor, or a sensing element, such as a resistive temperature detector, that is near or adjacent to the heating element. The swapping of the terminals may be facilitated using switching elements (e.g. a network of switching elements), such as switching elements comprising switching transistors operatively connected to the terminals of the resistor. For example, for a thermal sensor comprising two terminals, with the current flowing from the first terminal 1 to the second terminal, the opposite polarity current can be provided by using the same current source but changing the direction of the current (e.g. by biasing the thermal sensor in the opposite way), such that the current flows from the second terminal to the first terminal. The switching elements may be transistors which are configured such that their gate/control terminal can determine the direction of the current flow. The switching elements may be monolithically integrated, or may be provided externally.

In some implementations, the timing of the opposite polarity currents can be adjusted. For example, the on-time, off-time and/or delay between the pulses of the opposite polarity currents may be adapted as desired. Additionally or alternatively, the magnitude of the opposite polarity currents can also be adjusted, as desired.

The above operations of the flow sensor may be applied in multiple steps, e.g. by running the heater at several (different) temperature levels (given by different levels of power levels), to aid in the selectivity of different components of the fluid.

The fluid sensor may comprise one or more recessed regions within the first dielectric membrane and/or the second dielectric membrane. The recessed regions could be designed such that there are no recessed regions between the temperature sensing elements and the heating elements within a single membrane. Alternatively the recessed regions could be designed such that one of the temperature sensing elements is on one side of the recessed region, and another temperature sensing element and a heating element are on the same membrane is on the opposite side of the recessed region.

The recessed regions or discontinuities in the dielectric membrane provide an interruption (or partial interruption) in the thermal conduction path through the solid of the dielectric membrane. This in turn will mean that the heat path will occur more through the fluid above the recess (via conduction and convention) or through the cavity space formed as a result of the recess (mainly through fluid conduction). In both cases (heat above the cavity space or within the cavity space), the heat dissipation will depend on the thermal conductivity of the fluid. This increases the sensitivity of the differential signal to the thermal conductivity of the fluid as a larger percentage of heat loss will be through the fluid.

The one or more recessed region may comprise one or more discontinuous regions where the thickness of the dielectric membrane is discontinuous or varies from an average or most common dielectric membrane thickness.

The one or more recessed region may be located between the heating element and an edge of the dielectric membrane.

An edge of the dielectric membrane may refer to a perimeter edge of the dielectric membrane, in other words, the area where the dielectric membrane meets or joins the semiconductor substrate. The area of the dielectric region above the semiconductor substrate may refer to the area of the dielectric region outside the dielectric membrane.

The recessed regions may be holes (perforations) through the dielectric membrane. This would be advantageous, as the thermal conduction path through the solid of the dielectric membrane will be impeded and this will mean that the thermal conduction will occur through the holes (mainly via conduction) or above the holes (via both conduction and convection), thus facilitating the measurement of the composition of the fluid based on the different thermal conductivity of each of the components of the fluid. The holes may refer to apertures, perforations or slots extending through an entire height or depth or thickness of the dielectric membrane. This forms a fluid flow path and provides fluid connection between area above and area below membrane.

The at least one of the one or more holes may comprise an elongate slot extending towards opposite edges of the dielectric membrane. The elongate slot may not extend completely to the edges of the dielectric membrane or completely isolate the dielectric membrane either side of the elongate slot. The elongate slot increases thermal isolation across a width of the dielectric membrane of the device. Optionally the elongate slot may be extending in a same direction as one or more heating elements and/or sensing elements. The elongate slots may be, for example, rectangular, square, or semicircle.

The one or more holes may comprise an array of perforations. The perforations may comprise individual holes significantly smaller than a width of the dielectric membrane of the device. The array of perforations may can extend substantially across a width of the device.

The at least one recessed region may comprise a partial recess within the dielectric membrane. The partial recess or trench may extend from a top surface of the dielectric membrane or may extend from a bottom surface of the dielectric membrane. The partial recess may extend partially through a height or depth or thickness of the dielectric membrane. The at least one perforation may be in the form of a trench formed from the top or the bottom surface but not penetrating the other surface.

The discontinuities may be referred to as a gap in the membrane from the top surface to the bottom surface. Though, not as effective in terms of the thermal performance, a discontinuity could also refer to a trench or partial hole created from either the top or the bottom surface (if an upside-down membrane is used) without penetrating the other surface. The advantage of such partial holes is that they could impact less the mechanical strength of the membrane and in some cases they may be easier to be manufactured. Moreover, such partial holes could be used to hermetically seal the bottom side of the membrane or allow no fluid penetration below the membrane.

The at least one recessed region may have a meander shape. In other words, the discontinuity may have a non-standard shape such as a concertina or corrugated shape formed of a series of regular sinuous curves, bends, or meanders.

The location, shape and size of the recessed regions maybe identical on both the first and second dielectric membranes, or maybe different on the two dielectric membranes.

There may be one or more holes through the membrane to connect the upper side of the membrane to the lower side of the membrane via the fluid to be sensed. The one or more holes also disrupts the thermal conduction path through the solid dielectric membrane, forcing more heat to dissipate via convection and conduction through the environment. The presence of the one or more holes also helps to reduce the power consumption of the device (for the same heater temperature), because of the reduction in the heat conduction losses (through the solid membrane). Furthermore, the presence of the one or more holes allows for a lower thermal mass of the membrane thus reducing the time needed for the heater to heat up and cool down.

The one or more holes or recessed region may be used to enhance the sensitivity/selectivity to any fluid or component of the fluid (e.g. air with a concentration of CO₂) with a thermal conductivity that is different to that of a reference fluid or another component of the fluid (e.g. air).

An arrangement and specific design of different holes and different sensing elements is provided to enhance the sensitivity to any fluid or component of the fluid (e.g. air with a concentration of CO₂) with a thermal conductivity that is different to that of a reference fluid or another component of the fluid (e.g. air).

The arrangement of different holes or slots (or recessed regions) may be placed symmetrically around the heating element and the temperature sensing elements.

The heating element may be driven at more than one temperature, to increase the selectivity of the device. Gas thermal conductivity varies with temperature, and this variation is different dependent on the gas. In one drive mode, the heater can be driven at a temperature where the thermal conductivity of air and carbon dioxide are identical, and then used to detect another gas (e.g. hydrogen or methane). In this scenario, there will be no unwanted response from carbon dioxide and thus the selectivity of the device is improved. The heater can also be run at the temperature that provides the optimum sensitivity for the gas that is being measured.

The fluid sensor may comprise an array of multiple pairs of dielectric membranes located over multiple etched portions of the semiconductor substrate, each membrane pair having: at least two temperature sensing elements located within the dielectric membrane; a separate heating element, or one of the temperature sensing elements acting as a heating element. Each membrane pair may have its own output circuitry, or there may be a single output circuitry multiplexed across each membrane pair, or a combination maybe used. Each membrane pair may be operated at a different temperature. Each membrane pair maybe identical, or maybe different from the other membrane pairs.

The fluid sensor may further comprise a covering located on a surface of the sensor, where the covering may comprise a hole configured to allow fluid travel from an outer surface of the covering to a fluid channel above the first dielectric membrane.

According to a further aspect of the disclosure, there is provided a fluid sensor for sensing a concentration or composition of a fluid, the sensor comprising:
a semiconductor substrate comprising a first etched portion and a second etched portion;
a dielectric region located on the semiconductor substrate, wherein the dielectric region comprises a first dielectric membrane located over the first etched portion of the semiconductor substrate, and a second dielectric membrane located over the second etched portion of the semiconductor substrate;
two temperature sensing elements on or within the first dielectric membrane and two temperature sensing elements on or within the second dielectric membrane;
an output circuit configured to measure a differential signal between the two temperature sensing elements of the first dielectric membrane and the two temperature sensing elements of the second dielectric membrane;
wherein the first dielectric membrane forms a first sealed region that, in use, is exposed to the fluid via one or more holes; and
the second dielectric membrane forms a second sealed region, wherein the second sealed region is isolated from the fluid.

According to a further aspect of the disclosure, there is provided a method of testing a fluid sensor, wherein the second region comprises a sealed region and wherein the sealed region comprises a fluid of a first fluid composition, the method comprising:
placing the fluid sensor in a known atmosphere of a second fluid composition for a predetermined period of time, wherein the second fluid composition is different to the first fluid composition;
obtaining a differential signal between the first temperature sensing element and the second temperature sensing element after the predetermined period of time;
comparing the obtained differential signal to a predetermined expected signal, wherein the predetermined expected signal represents a differential signal expected when no fluid is exchanged between the sealed region and the known atmosphere;
determining whether a leak is present in the fluid sensor using the comparison between the obtained different signal and the predetermined expected signal.

According to a further aspect of the disclosure, there is provided a method of measuring a concentration or composition of a fluid using a fluid sensor as described above, the method comprising: applying an electrical bias to the heating element; and monitoring the electrical bias applied to the heating element and using the value of the electrical bias applied to the heating element and the temperature of the first temperature sensing element or the differential signal to determine the concentration or composition of the fluid based on thermal conductivity of the fluid.

Applying an electrical bias to the heating element may comprise applying an electrical bias such that the differential signal between the temperature sensing elements on the first dielectric membrane, and the temperature sensing elements on the second dielectric membrane may be minimised. Minimised may refer to reducing the differential signal to zero or substantially zero.

The electrical power, current, or voltage applied to the heating element may be adjusted to bring to zero or substantially zero the differential signal between the temperature sensing elements on the first dielectric membrane, and the temperature sensing elements on the second dielectric membrane (by varied the heating element power, current, or voltage could be such that the resistances of the temperature sensing elements or the voltages across the temperature detectors are equal). This may be done during the calibration of the sensor or during the operation of the sensor. This could be set as calibrated point, giving a zero differential signal. Alternatively, this could be set during the operation and the heater power/current/voltage could be measured as an indication of the fluid compositions or the concentration of its components The change in the electrical power, voltage or current through the heater may be monitored to measure one or more concentrations of specific components of the fluid based on their different thermal conductivities.

The measurement of the differential signal (for example, the differential resistance) can be performed in a number of ways. A first way is to connect the temperature sensing elements in two branches and applying a constant current to each branch. In the first branch there is a temperature sensing element from the first membrane connected to ground, and a temperature sensing element from the second membrane connected between the constant current source and the temperature sensing element from the first membrane. The second branch has a temperature sensing element from the second membrane connected to ground, and a temperature sensing element from the first membrane connected to the constant current source and the temperature sensing element from the second membrane. The voltage difference between the junction of the temperature sensing elements will give a differential signal based on the differences in temperatures of the two membranes and can be measured using a differential amplifier. A further method is to use a Wheatstone bridge or other type of bridges. For both these methods, a calibration can be done initially to set a zero-point value. This can either set a differential voltage value when the target fluid (or component of the target fluid) is not present, or modify the current to one of the resistors to ensure the differential voltage is at zero when the target fluid is not present. Alternatively, the calibration can be done initially to set a zero point value of the differential signal when the component of the fluid (e.g. CO₂) is known (e.g. 400 ppm of CO₂ in air) by using an external precision CO2 device (e.g. NDIR sensor).

The method may comprise driving the heating element in pulse mode or AC mode to modulate the temperature of the heating element to vary the differential signal; and using the differential signal to selectively differentiate between different fluid components and/or determine the concentration of the different components. In implementations, this may comprise heating the sensor to a first temperature where the thermal conductivity of air and the thermal conductivity of the target gas (for example carbon dioxide) are the same. This then facilitates the determination of the effect of other gases in the air (such as water vapour). The sensor may then be operated at a second (different) temperature, and the effect of other gases may be accounted for (i.e. reduced or cancelled) using e.g. a look up table or a formula, so that only the effect of the target gas is determined. The formula and/or look up table may be predetermined for the target gas. The device may therefore comprise circuitry or a control system facilitating temperature modulation of the sensor using e.g. the heating element(s) or other suitable heater.

In implementations, the thermal conductivity fluid sensor can be used in a mode where, instead of a constant DC temperature, the temperature is varied.

Thermal conductivity sensors generally work at a constant (DC) operating temperature and measure the heat loss to the surrounding medium. This DC method is typically most effective when there is a single known gas (such as hydrogen) and the signal can be directly related to the concentration of the known gas. However, if the gas type is unknown then the concentration of the target gas cannot be determined using this method. Moreover, if another gas is present as well as the known target gas, then again the gas concentration cannot be determined. That means it is not possible to determine the concentration of the target gas in a mixture (e.g. H₂ and He) or where the target gas is an unknown gas.

However, if the temperature of the thermal conductivity sensor is changed, then the thermal time constant associated with the transition depends not only upon the thermal conductivity λ of the gas, but also on the specific heat capacity c of the gas (and the density of the gas ρ). The equation for the thermal time constant is:

$${τ}_{th} = \frac{ρ cV}{λ A}$$

Where τₜₕ is the thermal time constant, V is the volume of a body and A is surface area of the body.

As such, by varying the temperature of the sensor, the differences in density and heat capacity can be used to distinguish between different gases, in addition to the differences in the thermal conductivity.

The temperature of the heating element may be modulated by varying the current, voltage or power to different levels and/or with different electrical pulses in order to selectively differentiate between different fluid components and/or to provide information regarding the concentration of such components.

The temperature of the heater may be modulated and the output from the read out circuit comprising the temperature sensing elements from the first and second dielectric membranes at different temperatures may be assessed against reference values, and the difference between the two may be indicative of the flow composition.

The heating element temperature may be modulated by applying different power levels to increase sensitivity and selectivity to different fluid components based on their thermal conductivity variation with temperature. For example, the difference between the thermal conductivities of CO₂ and the air is higher at room temperature than at high temperatures. The opposite is true for Methane, so the difference between the thermal conductivities of methane and the air is lower at room temperature than at high temperatures. Hydrogen has also a different variation of the thermal conductivity with temperature than that of CO₂ or air. By running the heater at different temperature levels (i.e. modulating the temperature of the heater), it is entirely possible to differentiate between the contributions of different concentrations of fluid components in the fluid. In this way, for example, Hydrogen and CO₂ contributions can be decoupled and their concentration values can be found.

The heater (also referred to as the heating element) may be operated in a pulse mode (e.g. driven with a square wave, sinusoidal wave, Pulse Width Modulated wave (PWM), Pulse Density Modulation, etc.) or continuous mode. The pulse mode has, among others, the advantage of reduced power consumption, reduced electromigration for enhanced device reliability/lifetime, and improved fluid properties sensing capabilities. Pulses could be used in different polarities to further reduce the impact of electromigration on the heating element.

Different drive modes and measurement modes are possible. For example, the heater can be driven using PWM, and the off time of the PWM can be used to measure heater resistance, and/or differential signal. This measurement can be done in a very short time, faster than the thermal time constant of the membrane to avoid self-heating.

Selectively differentiating between different fluid components and/or determining the concentration of the different components may comprise using a neural network.

An algorithm containing machine learning and artificial intelligence may be implemented. For example, the sensor or a fluid sensing system may further comprise a controller or a processing system comprising a neural network. The neural network may be trained using data from different known gases or mixture of gases at different temperatures. The use of a trained neural network to identify known gases or a mixture of gases can improve accuracy, sensitivity and selectivity of the fluid sensor.

The neural network may be trained to recognise the composition of a gas mixture based on the differential signal between the temperature sensing elements of the first and second dielectric membranes. The neural network could be trained using supervised learning based on a set of data of sensor output values for known gas mixtures at a set of heating element temperatures. The inputs to the neural network could be the sensor output values at a predetermined set of temperatures. The neural network may be configured to process each differential signal from the temperature sensing elements of the first and second dielectric membranes in order to determine the components of the gas mixture and the concentrations of each component in the gas mixture. The outputs from the neural network could be the fraction of each gas in the mixture. Synthetic training data could be generated to enhance the training by providing, for example, many more combinations of gases than would be practically realisable in a real laboratory. A support-vector machine could be trained to discriminate between different gases.

The method may comprise: applying a modulated function to the heating element, the temperature sensing elements of the first and second dielectric membranes; measuring the modulation, the time delay, or the phase shift of the temperature signal from the first temperature sensing element or the differential signal between the temperature sensing elements of the first and second dielectric membranes; and determining a concentration or composition of the fluid using the measured modulation, time delay or phase shift.

A transient, modulated, or pulsed signal may be applied to either the heater element or the temperature sensing elements of the first and second dielectric membranes, and the signals from the first or second temperature detectors will consequently be transient, and their time shape, time delay, or phase shift depends on both the thermal conductivity and the thermal diffusivity of the fluid around the sensor and its concentration of particular fluid components with different thermal conductivities and the thermal diffusivities

The heaters or the temperature sensing elements of the first and second dielectric membranes can be biased with a transient signal (e.g. AC, square wave, pulsed, step). Using transient based signals, the thermal diffusivity can be determined using the measured values from the first and second temperature sensing elements. In this way, more information can be extracted from the environment.

In a method of transient fluid sensor drive modes, a step change in input current can be applied to the heater and the time constant for the temperature rise in the heater can be measured. This time constant can give information about the thermal conductivity and diffusivity of the environment. Both can be used to identify gas concentration.

In another method of transient sensor drive modes, a sinusoidal wave can be applied to the heater. The change in amplitude and change in phase shift can provide information on thermal conductivity and thermal diffusivity, thus providing information on the gas concentration.

Additionally or alternatively, the heating element(s) may be provided with a DC bias point onto which a small AC signal (such as e.g. an AC, square wave, pulsed or step signal) can be superimposed. By using small AC based signals, the thermal diffusivity, conductivity and/or thermal capacity of the target fluid can be determined using the measured values from the temperature sensing elements of the first and second dielectric material. The changes in the amplitude, phase shift and/or changes in frequency of the measured values can provide information on thermal conductivity and/or thermal diffusivity, thus providing information on the gas concentration, or facilitating the selection between different components of the gas.

Any of the resistive temperature detectors may be driven in short pulses of power, voltage or current. The temperature sensing elements (resistive temperature detectors) may be driven in a pulse mode (e.g. driven with a square wave, sinusoidal wave, Pulse Width Modulated wave, Pulse Density Modulation, etc.) or continuous mode. The pulse mode has, among others, the advantage of reduced self-heating of the temperature sensing elements, which minimises the noise and increases the sensitivity or the signal to noise ratio.

Whilst several methods are described, any other method of driving the sensor that can provide information on the environment that is being measured may be used.

According to a further aspect of the present disclosure, there is provided a fluid sensing system comprising a fluid sensor as described above; and a controller configured to perform a method as described above.

The fluid sensing system may include a hardware or software interface wherein an algorithm is implemented to facilitate to selectively differentiate between different fluid components and/or to provide information regarding the concentration of such components.

A software algorithm configured to perform any of the methods as described above could be implemented to differentiate between these components and increase sensitivity related to each of the components of the fluids. The software algorithm could be implemented in a local microprocessor. Calibrated data could be stored in a memory device or integrated circuit. Alternatively, the software could be incorporated within an ASIC and driving of the sensor and processing of the signal could be done within an ASIC.

Processing of the signal could also be done remotely in a sensor hub, or on an external server accessed using the Internet (for example, the cloud).

Sampling and averaging of the data, as well as ways to remove outliers from the data could also be used as part of an algorithm and could be implemented in hardware using different electronic components such as micro-controllers, memories or could be done using an ASIC.

Readings from the sensor may be averaged in several ways, for example using a moving mean average or a moving median average. A moving mean average is useful for removing random noise from the signal. A moving median average is useful for removing outliers.

Algorithms may also be used to account for drift in the sensor, in particular to account for drift in a partially hermetic sealed reference due to an outside atmosphere affecting the sealed region over time. One method is automatic based line calibration. In this method the sensor system may assume that over a given time period (for example, 24 hours) the sensor has been exposed at least once to a nominal concentration of the target gas. For example, in the case of a carbon dioxide sensor—this method may assume that at least once in 24 hours the sensor is exposed to fresh air with a carbon dioxide concentration of 400 ppm. This facilitates a self-calibration by using the lowest reading seen by the sensor over the 24 hour period, under the assumption that this lowest reading corresponds to the exposure to the nominal concentration of the target gas (e.g. fresh air of the above example). The chosen time period can be any suitable time period, and may be less or more than 24 hours. The time period may be always fixed, or may change based on e.g. the sensor readings or over the sensor lifetime.

Additionally or alternatively, a further method to reduce drift in the sensor with a partially hermetic sealed reference comprises to estimate the effect of the outside atmosphere to the sealed region. This may comprise storing an initial value in a variable, and updating the variable over time based on the sensor readings. The variable may also be updated based on the value of the variable. The variable represents a value that corresponds to the sealed region atmosphere. This variable may then subsequently be used to determine a more accurate value of the gas concentration. More than one variable can also be used in a variety of configurations.

According to a further aspect of the disclosure, there is provided a method of manufacturing a fluid sensor for sensing a concentration or composition of a fluid, the method comprising:
forming a semiconductor substrate comprising a first substrate portion and a second substrate portion, the first substrate portion comprising a first etched portion and the second substrate portion comprising a second etched portion;
forming a dielectric region located on the semiconductor substrate, wherein the dielectric region comprises a first dielectric membrane provided on the first etched portion and a second dielectric membrane provided on the second etched portion;
forming a first temperature sensing element located on or within the first dielectric membrane and a second temperature sensing element located on or within the second dielectric membrane;
forming an output circuit configured to measure a differential signal between the first temperature sensing element and the second temperature sensing element,
   wherein, the fluid sensor comprises a first region configured to be exposed to the fluid, and a second region configured to be isolated from the fluid,
   wherein the first dielectric membrane is located in the first region, such that in use, the first dielectric membrane is exposed to the fluid, and
   wherein the second dielectric membrane is located in the second region such that in use, the second dielectric membrane is isolated from the fluid.

### BRIEF DESCRIPTION OF THE FIGURES

Some embodiments of the disclosure will now be described by way of example only and with reference to the accompanying drawings, in which:

FIG. 1 shows a top view of a thermal conductivity fluid sensor with two membranes, a heating element on each membrane, two temperature sensing elements on each membrane and two temperature sensing elements outside the membrane alongside each membrane;

FIG. 2 shows a cross-section of a thermal conductivity fluid sensor with two membranes, where one of the membranes is isolated from the environment;

FIG. 3 shows the cross-section of a thermal conductivity fluid sensor with two membranes formed by a front side etch, where one of the membranes is isolated from the environment;

FIG. 4 shows the cross-section of a thermal conductivity fluid sensor with two membranes formed by an isotropic front side etch, where one of the membranes is isolated from the environment;

FIG. 5 shows the cross-section of a thermal conductivity fluid sensor with two membranes formed by a timed front side etch, where one of the membranes is isolated from the environment;

FIG. 6 shows the cross-section of a thermal conductivity fluid sensor with two membranes packaged in a flip chip method;

FIG. 7 shows the cross-section of a thermal conductivity fluid sensor with two membranes packaged in a flip chip method where the membrane exposed to the environment has an encapsulation with holes;

FIG. 8 shows a top view of a thermal conductivity fluid sensor with two membranes, a heating element on each membrane, two temperature sensing elements on each membrane and an ambient temperature sensor outside the membranes;

FIG. 9 shows a top view of a thermal conductivity fluid sensor with two membranes, a heating element on each membrane, two temperature sensing elements on each membrane and an ambient temperature sensor outside the membranes with no slots on the membranes;

FIG. 10 shows a top view of a thermal conductivity fluid sensor with two membranes, a heating element on each membrane, two temperature sensing elements on each membrane and an ambient temperature sensor outside the membranes with an array of holes on the membranes;

FIG. 11 shows a top view of a thermal conductivity fluid sensor with two membranes, a heating element on each membrane, two temperature sensing elements on each membrane and an ambient temperature sensor outside the membranes, where the temperature sensing elements within each membrane are different;

FIG. 12 shows a circuit diagram of a thermal conductivity sensor comprising two Wheatstone bridges;

FIG. 13 shows a circuit diagram of a thermal conductivity sensor comprising a single Wheatstone bridge;

FIG. 14 shows a circuit diagram of a thermal conductivity sensor where a balance voltage can be used to balance the Wheatstone bridge;

FIG. 15 shows a circuit diagram of a thermal conductivity sensor with a feedback circuit to control the heater;

FIG. 16 shows the top view of a thermal conductivity fluid sensor with two membranes and two resistors on each membrane;

FIG. 17 shows the circuit for a thermal conductivity sensor where there are two resistors on each membrane acting as heating elements and temperature sensing elements;

FIGS. 18 shows the circuit for a thermal conductivity sensor where there are two temperature sensing elements on each membrane, and one of the temperature sensing elements on each membrane can be switched to also operate as a heating element;

FIG. 19 shows the circuit for a thermal conductivity sensor where the voltage on one of the branches of the Wheatstone bridge is controlled by a feedback circuit;

FIGS. 20 shows the circuit for a thermal conductivity sensor where the voltage on one of the branches of the Wheatstone bridge is controlled by a feedback circuit which also takes input from an external sensor;

FIG. 21 shows the top view of a thermal conductivity sensor where there are two membranes and each membrane has two diodes that are temperature sensing elements;

FIG. 22 shows the top view of a thermal conductivity sensor where there are two membranes and each membrane has two temperature sensing elements, one of which is a resistor and one is a diode;

FIG. 23 shows the circuit for a thermal conductivity sensor where the temperature sensing elements are diodes;

FIG. 24 shows the circuit for a thermal conductivity sensor where the temperature sensing elements are diodes and resistors;

FIG. 25 shows the top view of a thermal conductivity fluid sensor where there are two pairs of membranes;

FIG. 26 shows the cross-section of a thermal conductivity fluid sensor package with a sealed region and an exposed region, and two sensor chips and an ASIC chip;

FIG. 27 shows the cross-section of a thermal conductivity fluid sensor package with a sealed region and an exposed region, and one sensor chip and an ASIC chip;

FIG. 28 shows the cross-section of a thermal conductivity fluid sensor package with a sealed region and an exposed region, and two sensor chips and an ASIC chip with one sensor chip stacked on the ASIC chip;

FIG. 29 shows the cross-section of a thermal conductivity fluid sensor package with a sealed region and an exposed region, and two sensor chips and an ASIC chip with one sensor chip stacked on the ASIC chip, and the sensor chips attached in a flip chip method;

FIG. 30 shows the cross-section of a thermal conductivity fluid sensor package in a chip scale package with the ASIC chip as part of the package;

FIG. 31 shows the cross-section of a thermal conductivity fluid sensor with two ambient temperature sensors in the sealed region, and two in the exposed region;

FIG. 32 show an alternate circuit for a thermal conductivity fluid sensor where the ambient temperature sensors are also in the bridge;

FIG. 33 shows an alternate circuitry for a thermal conductivity fluid sensor where the read out circuit also measures two ambient temperature sensors;

FIG. 34 shows an alternate circuitry for a thermal conductivity fluid sensor where the heater also acts as a temperature sensor on the membrane, and the ambient temperature sensors are also measured in a bridge;

FIG. 35 shows an alternate circuitry for a thermal conductivity fluid sensor where the differential signals from the on-membrane and off-membrane temperature sensing elements go through a further differential amplifier;

FIG. 36 shows a flow diagram of a method to better improve the sensitivity of the measured signal;

FIG. 37 shows a 3D schematic of a thermal conductivity fluid sensor.

FIG. 38 shows the top view and cross-section of a thermal conductivity fluid sensor comprising two sealed cavities, with one containing holes.

FIG. 39 shows a graph with thermal conductivities of air and different gases at different temperatures.

FIG. 40 shows an alternate circuit diagram for a thermal conductivity fluid sensor.

FIG. 40a shows the current through the heater for the circuit in FIG. 38.

FIG. 41 shows an alternate circuit diagram for a thermal conductivity fluid sensor.

FIG. 41a shows the current through the heater and the voltage across it, as well as the reading computation for the circuit in FIG. 43.

FIGS. 42 and 43 show alternate circuit diagrams for a thermal conductivity fluid sensor based on FIG. 41a.

FIG. 44 shows a comparison between DC and current-reversal DC voltage measurements.

FIG. 45 shows a circuit schematic for driving the sensors in who different current directions.

FIG. 46 shows a table of thermal properties of different gases.

FIG. 47 shows a top view of a thermal conductivity fluid sensor with two membranes and two temperature sensors on each membrane. The temperature sensors are connected together in a bridge configuration located on the sensor chip.

FIG. 48 shows a circuit diagram of a Wheatstone bridge driven in constant current.

FIG. 49 shows a cross-section schematic of a thermal conductivity fluid sensor with an additional layer between the dielectric layer and the substrate, provided with a cavity portion.

FIG. 50 shows schematically a cross-section of a further thermal conductivity fluid sensor having a fluid sensitive material and electrodes connected to the fluid sensitive material.

FIG. 51 shows a flow chart of a method for testing a thermal conductivity fluid sensor as described herein.

FIG. 52 shows schematically a cross-section of a thermal conductivity fluid sensor having three dielectric membranes. In use, a first reference membrane is sealed from the atmosphere, a second membrane has holes and is exposed to the atmosphere, and a third reference membrane is exposed to the atmosphere from a top surface of the membrane, but is sealed on a lower surface of the membrane.

FIG. 53 shows schematically a cross-section of a thermal conductivity fluid sensor having three dielectric membranes. In use, first and second reference membranes are sealed from the atmosphere, and a third membrane is exposed to the atmosphere.

### DETAILED DESCRIPTION OF THE PREFERRED EMBODIMENTS

Some examples of the disclosed device are given in the accompanying figures.

FIGS. 1 & 2 show the top view and cross-section of a thermal conductivity fluid sensor. It comprises a chip 1 made of a semiconductor substrate 11 and a dielectric layer 10, with a first dielectric membrane 4, and a second dielectric membrane 4A suspended over etched portions of the semiconductor substrate. The first dielectric membrane 4 has a heating element 2 and two resistive temperature sensing elements 8 and 8A. Tracks 7 provide electrical connection from the bond pads to the heating element 2. There are also recessed regions 12 in the shape of slots within the membrane. Similarly the second dielectric membrane 4A has a heating element 2A and two resistive temperature sensing elements 8B and 8C, and recessed regions 12 in the shape of slots. There are a further 4 temperature sensing elements 3, 3A, 3B, 3C on the chip. The chip is attached to a base 101, and a cap 110 is placed over the second dielectric membrane 4A resulting in a sealed region 200, where the fluid (typically but not limited to a gas) is trapped, and the composition of the trapped fluid 200 doesn't change with the change in fluid or environment around the sensor. The first dielectric membrane 4 is exposed to the environment and the thermal behaviour of the membrane changes with change in fluid around the sensor—for example is the fluid composition changes to change the thermal conductivity of the fluid. Hence the power consumption required by the heating element 2 on membrane 4 is affected by change of composition of the surrounding fluid, while the power consumption required by the heating element 2A in membrane 4A does not change.

The membranes 4, 4A are formed by back side etching using DRIE resulting near vertical sidewalls.

For example if the encapsulated fluid 200 is air, and the surrounding fluid is also air, then if the concentration of carbon dioxide in the surround fluid/air increases, the overall thermal conductivity of the surrounding fluid will decrease as the thermal conductivity of carbon dioxide is smaller than air. In that case the heating element 2 on membrane 4 will have slightly lower thermal losses as compared to heating element 2A on membrane 4A. So heating element 2 will require slightly less power to maintain membrane 4 at a target temperature as compared to the power required by heating element 2A to maintain membrane 4A at the same temperature. Alternately if the same bias (e.g. current, voltage, power) is applied to both heating elements 2,2A, then membrane 4 will reach a slightly higher temperature than membrane 4A.

Circuitry is used to drive both the membranes to a high temperature. An output circuit uses at least the temperature sensing elements 8, 8A, 8B, 8C to determine the change in fluid composition or the target gas. The circuitry might drive both the heaters 2,2A in a constant bias current, voltage or power. In this case the temperature of membrane 4A will stay the same if there is a change in fluid composition, but the temperature of membrane 4 will change. Using a differential output circuit such as a Wheatstone bridge will then give an output based on change in the fluid composition. Using two temperature sensors from each membrane rather than a single temperature sensor from each membrane in the Wheatstone bridge means that the sensitivity is doubled as it becomes a half bridge rather than a quarter bridge. Other drive methods can also be used to keep the Wheatstone bridge outputting zero volts while adjusting the bias on one of the heating elements 2,2A, and using the bias required to keep the zero output from the Wheatstone bridge to determine the fluid composition.

The encapsulated fluid 200 can be air, dry air, synthetic air, an inert gas such as nitrogen or argon. Alternately 200 could be a vacuum or a partial vacuum.

Circuitry to measure the sensor can also comprise two Wheatstone bridges, the first Wheatstone bridge comprising temperature sensing elements 8, 8A, 3, 3A, and the second Wheatstone bridge comprising temperature sensing elements 8B, 8C, 3B, 3C.

In this figure the heating elements are shown as wire heaters, but can be any other shape such as meander, ring, multi ring, circular etc. Similarly the temperature sensing elements can be any shape as well. In this figure the temperature sensing elements and the heating elements are all made from the same material layer and laterally spaced from each other. But they can also be made in different layers, and of different materials, and be either laterally or vertically spaced from each other, and can also be vertically stacked, or a combination. The resistors maybe made of a CMOS metal such as aluminium, tungsten, titanium or copper, or a non-CMOS metal such as gold or platinum, or from polysilicon or single crystal silicon. Similarly the membranes are shown as square with rounded corners, but can be square, rectangle or circular.

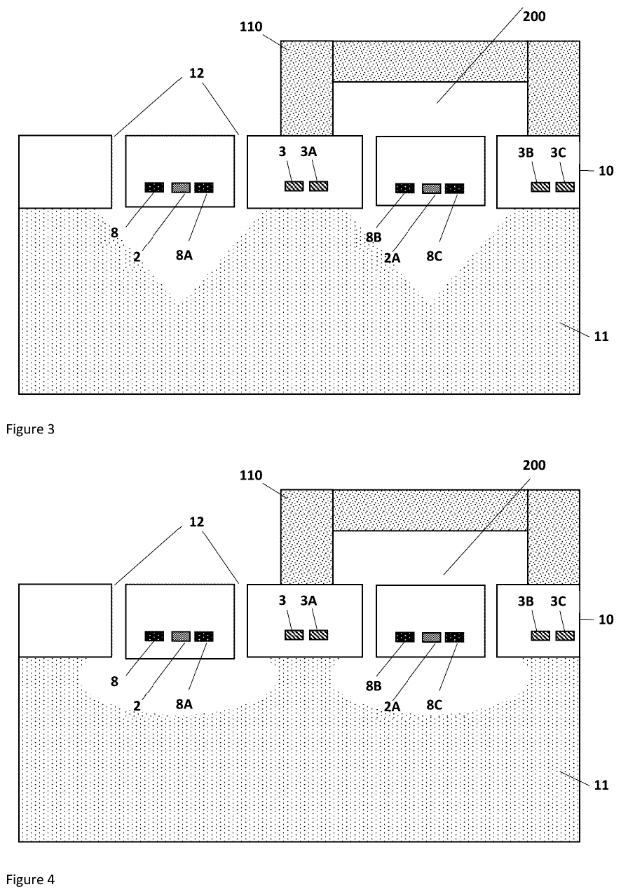

FIG. 3 shows the cross-section of an alternate thermal conductivity fluid sensor where the membranes 4,4A are formed by a front side anisotropic etch such as KOH or TMAH. In this case the etched portions of the substrates do not extend all the way to the bottom of the substrate, rather they stop at the crystal planes of the substrate. Such an etching usually results in a suspended membrane or micro-bridge, which not supported by the substrate along its entire perimeter, but rather is suspended by one or more beams.

FIG. 4 shows the cross-section of an alternate thermal conductivity fluid sensor where the membrane 4,4A are formed by a front side isotropic etch which results in curved surface of the substrate etched portion.

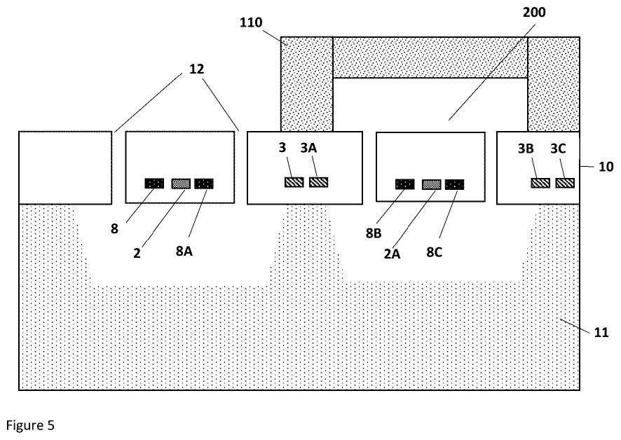

FIG. 5 shows the cross-section of a thermal conductivity fluid sensor where the membrane is created by a front side anisotropic etch, but the etch process is a timed etch, so as to result in a trapezoid shaped cavity or etched portion within the substrate.

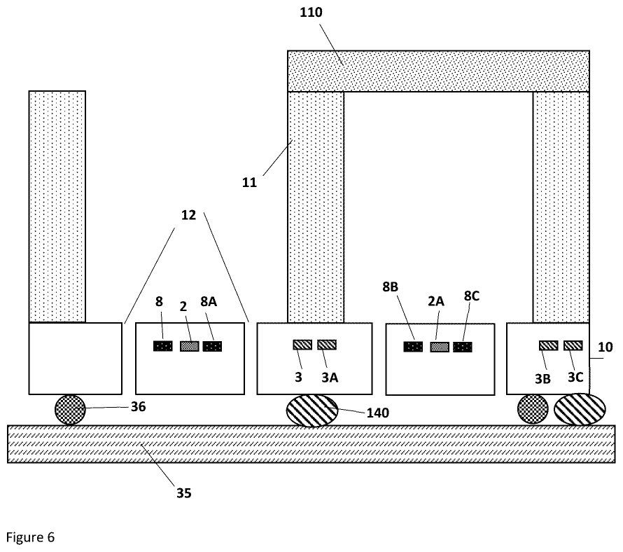

FIG. 6 shows the cross-section of a thermal conductivity fluid sensor where the chip is packaged in a flip-chip method. In this case the chip is attached to a PCB 35, and electrically connected by the use of solder balls 36. Membrane 4A is sealed by a lid 110 to seal the top, and also a sealant 140 to seal the fluid between the membrane and the PCB. The sealant 140 can be a polymer. It can also be a mixture of components and materials such as a rubber/glass/metal ring coated with a polymer.

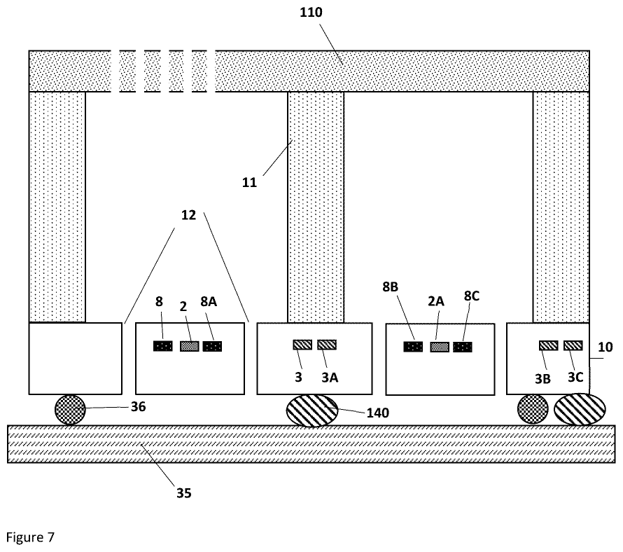

FIG. 7 shows the cross-section of another example of a thermal conductivity fluid sensor in a flip-chip package, where the lid 110 extends to the cavity portion of the exposed membrane as well, but there are holes in that portion of the lid to allow exchange with the surrounding fluid. The holes can help protect the membrane during handling, and also dust or moisture. Additional filters may also be added to protect against particles or moisture.

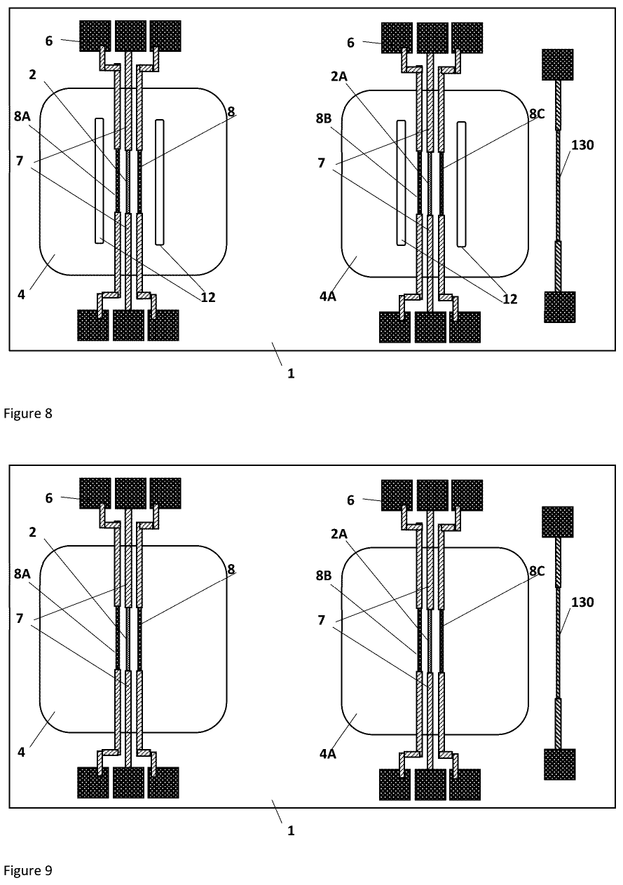

FIG. 8 shows the top view of another example of a thermal conductivity fluid sensor there is only one temperature sensing element 130 outside the membrane. The temperature sensing element 130 is used to measure the ambient temperature and helps provide a temperature compensation to the measured value of the fluid composition. The temperature sensing element shown in this example is a resistive temperature sensor, but can also be a diode, transistor a VPTAT or IPTAT circuit.

FIG. 9 shows the top view of another example of a thermal conductivity fluid sensor where there are no recessed regions within the membrane.

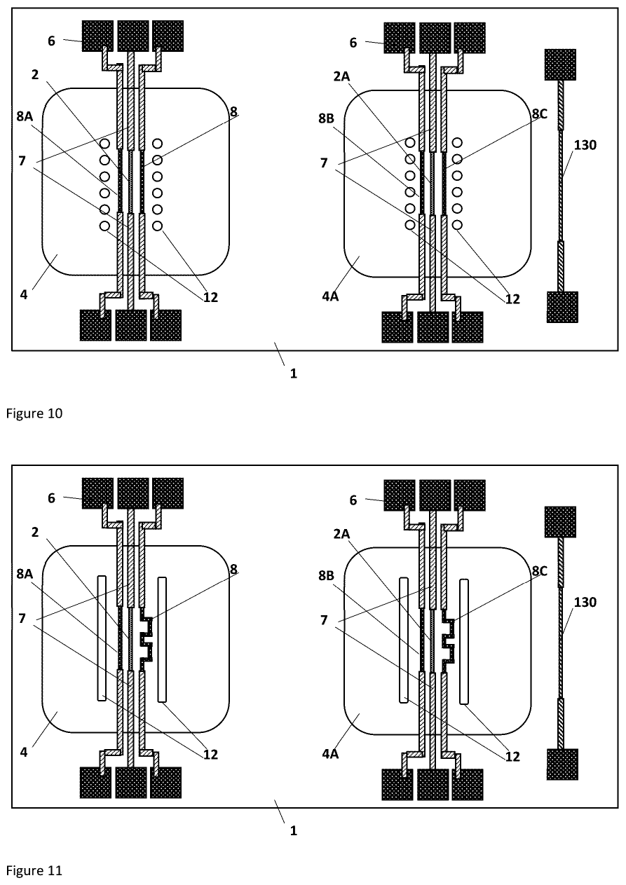

FIG. 10 shows the top view of another example of a thermal conductivity fluid sensor where the recessed regions 12 are an array of holes rather than slots. It should be noted that while FIGS. 8 and 10 show two examples or recessed regions many other shapes and sizes of recessed regions are possible. The recessed regions can also be in different locations on the membrane.

FIG. 11 shows the top view of another example of a thermal conductivity fluid sensor where the temperature sensing elements 8 and 8A have a different shape and resistance value. Similarly temperature sensing elements 8B and 8C are also different. But element 8 is identical to element 8C and element 8A is identical to element 8B. In this case although the temperature sensing elements within membrane 4 are different, they are identical to the corresponding temperature sensing elements of membrane 4A. The output circuit can still measure the differential signal across these temperature sensing elements as they are identical in the different membranes.

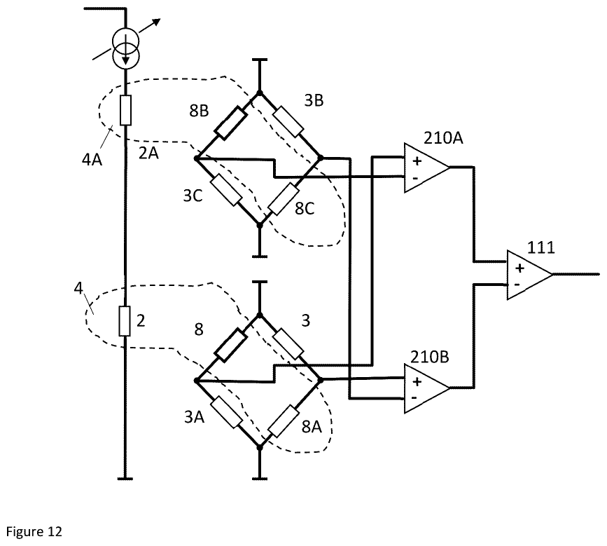

FIG. 12 shows circuitry for driving the thermal conductivity fluid sensor and measuring the output from the thermal conductivity fluid sensor shown in FIG. 1. Heating elements 2, 2A are both driven by a constant current source. The output circuit comprises two Wheatstone bridges. One bridge comprises temperature sensing elements 8, 8A from the first membrane, and temperature sensing elements 3,3A. The other bridge comprises temperature sensing elements 8B, 8C from the second membrane. Instrumentation amplifier 210A gets inputs from the left branch of both the Wheatstone bridges, while instrumentation amplifier 210B get inputs from the right branches. These then feed into instrumentation amplifier 111.

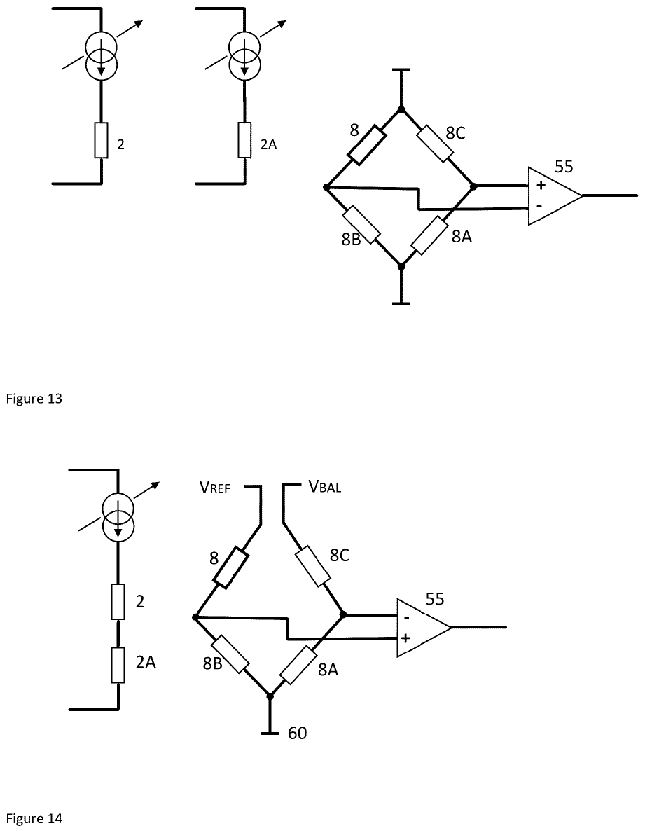

FIG. 13 shows circuitry for driving the thermal conductivity fluid sensor and measuring the output from the thermal conductivity fluid sensor shown in FIG. 8. Heating elements 2, 2A are driven by constant current sources. The output circuit comprises a Wheatstone bridge with temperature sensing elements 8,8A, 8B, 8C, and an instrumentation amplifier 55. If an identical current source is applied to both heating elements 2, 2A, then in normal conditions the temperature of both the membrane 4,4A will be the same, resulting identical resistance of the temperature sensing elements 8,8A, 8B, 8C, giving a zero-volt output on the output circuit. However if the surround fluid changes, for example to have a higher concentration of carbon dioxide, then the thermal conductivity of the surround fluid will decrease and power losses from membrane 4 will decrease, resulting in a slightly higher temperature than membrane 4A. In this case the temperature sensing elements 8,8A will have a slightly higher resistance than temperature sensing element 86,8C, resulting in a misbalance in the Wheatstone bridge and the output circuitry will give a non-zero output that can be used to determine the concentration of carbon dioxide present.

FIG. 14 shows another example of circuitry to drive and measure the output from the thermal conductivity fluid sensor. Except in this case both arms of the bridge have a different bias voltage. One arm is kept at a constant voltage of Vref, while the voltage to the other arm Vbal can be varied. This can serve many purposes, the main one being in calibration. Due to manufacturing tolerances there may be some mismatch between the resistive temperature sensors 8, 8A, 8B, 8C and when Vref and Vbal are equal the bridge might still give a non-zero output in normal conditions. So during calibration in a known environment, Vbal is adjusted until the output becomes zero. This value of Vbal is stored, and is also applied when making a measurement.

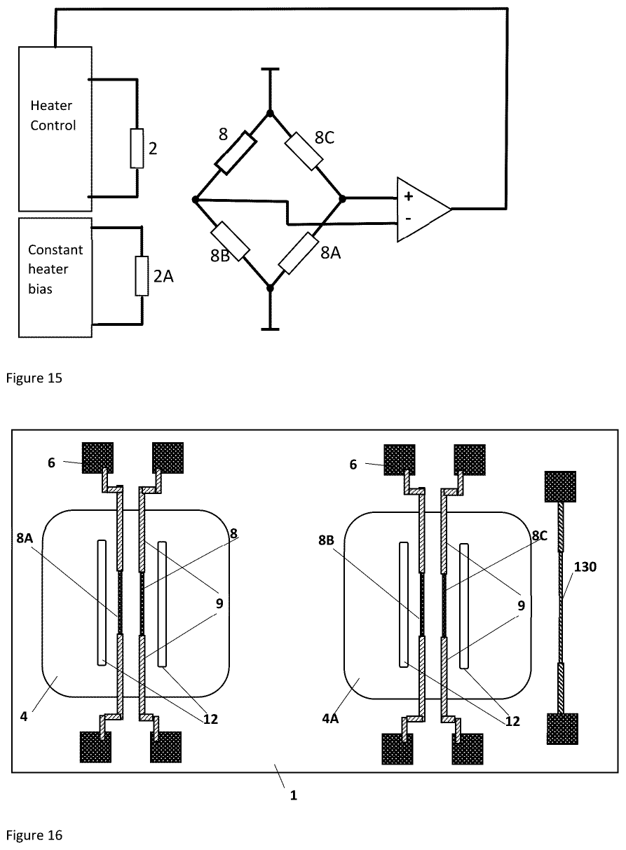

FIG. 15 show another example of circuitry to drive and measure the output from the thermal conductivity fluid sensor. In this case there is always a constant bias applied to the heating 2A, but the heating element 2 is controlled by a feedback loop from the Wheatstone bridge and amplifier circuit. In this case a heater control circuit varies the bias to heating element 2 until the output from the Wheatstone bridge and amplifier is zero, and the bias needed to drive the heating element is then used to determine the composition of the surrounding fluid.

FIG. 16 shows the top view of another example of a thermal conductivity fluid sensor comprising two membranes 4, 4A with two temperature sensing elements 8, 8A on membrane 4, and two temperature sensing elements 8B, 8C on membrane 4A. Unlike FIG. 8, there is no additional heating element on either of the membranes, instead one, or both of the temperature sensing elements can be used as the heating element.

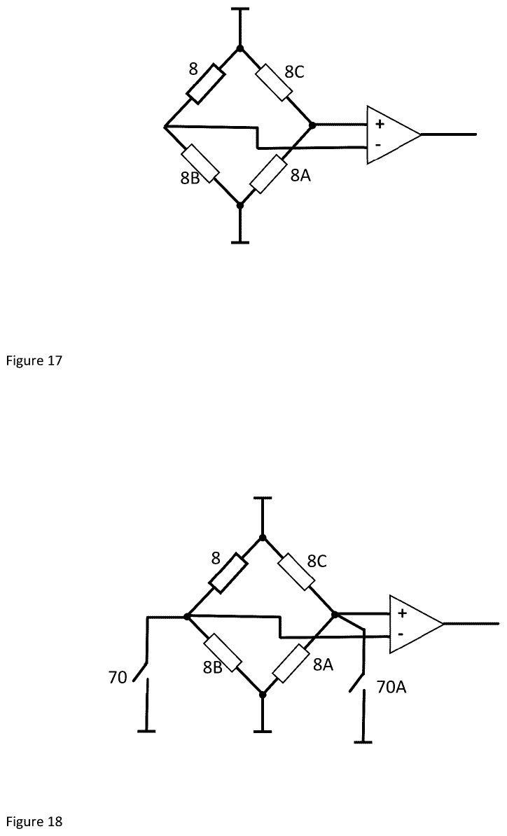

FIGS. 17 to 20 give some examples of circuits to drive and measure the output from the thermal conductivity fluid sensor. In FIG. 17 both the temperature sensing elements on both the membranes, 8, 8A, 8B, 8C all act as heating elements as well. A bridge circuit incorporates all four elements, and the voltage bias on the Wheatstone bridge also provides the bias for heating up the membranes, and also allows a differential measurement between them.

In FIG. 18 the temperature sensing elements 8 and 8C both act as heating elements as well as temperature sensing elements. To operate as heaters switches 70 and 70A are closed, connecting one of the terminal of elements 8, 8C directly to ground. When a measurement is to be made then switches 70, 70A are opened to connect element 8A and 8B as well, completing the bridge. The voltage bias may be lower during the measurement time. The measurement time should be short compared to the thermal time constant of the membranes so that the measurement does not affect the temperature.

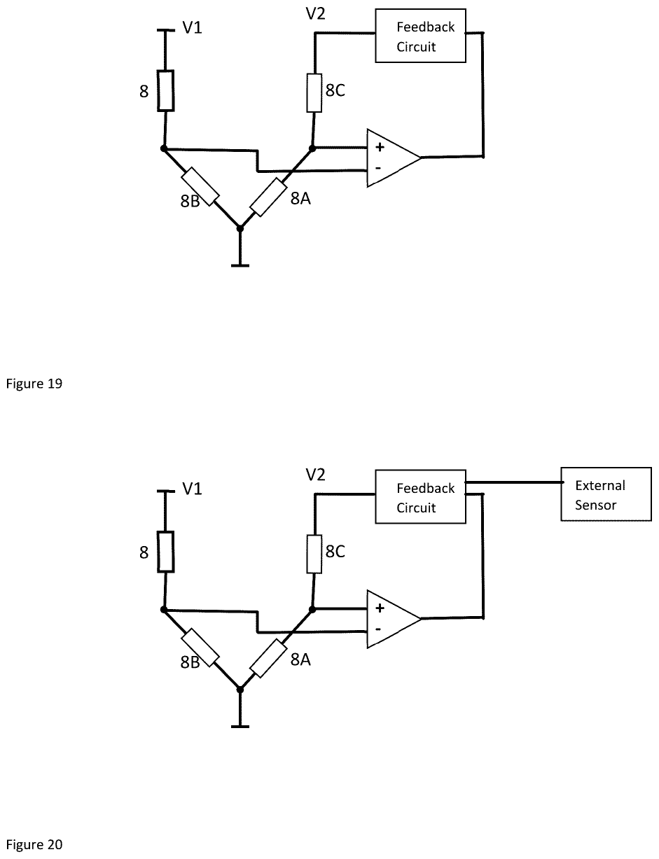

In FIG. 19 all four elements are used as both heaters and temperature sensing elements, but different voltages can be applied to each branch of the Wheatstone bridge. Voltage V1 is kept constant while a feedback circuit is used to vary V2. Such a circuit can be used in two ways. In one method the feedback circuit is used in calibration to adjust V2 until the amplifier gives a zero-volt output in a known environment, and then the same value of V2 is used always during measurement. In the second method the feedback circuit always varies the value of V2 until the output from the amplifier is zero, and the value of V2 required is used to determine the composition of the fluid.

FIG. 20 shows another circuit example which is similar to FIG. 19, except the feedback circuit may also use data from one or more external sensors. For example an external measurement of humidity, pressure or temperature can be used to apply an adjustment to the voltage V2 as part of compensating for signal changes due to these factors.

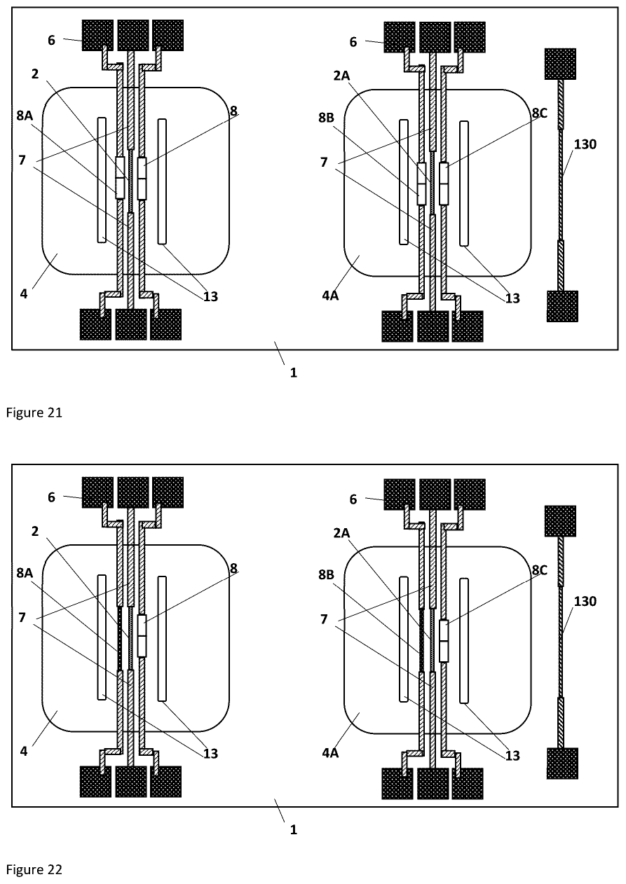

FIG. 21 shows the top view of a thermal conductivity fluid sensor where the temperature sensing elements 8, 8A, 8B, 8C are diodes instead of resistors. FIG. 22 shows an example where one of the temperature sensing element on each membrane is a resistor and one temperature sensing element is a diode.

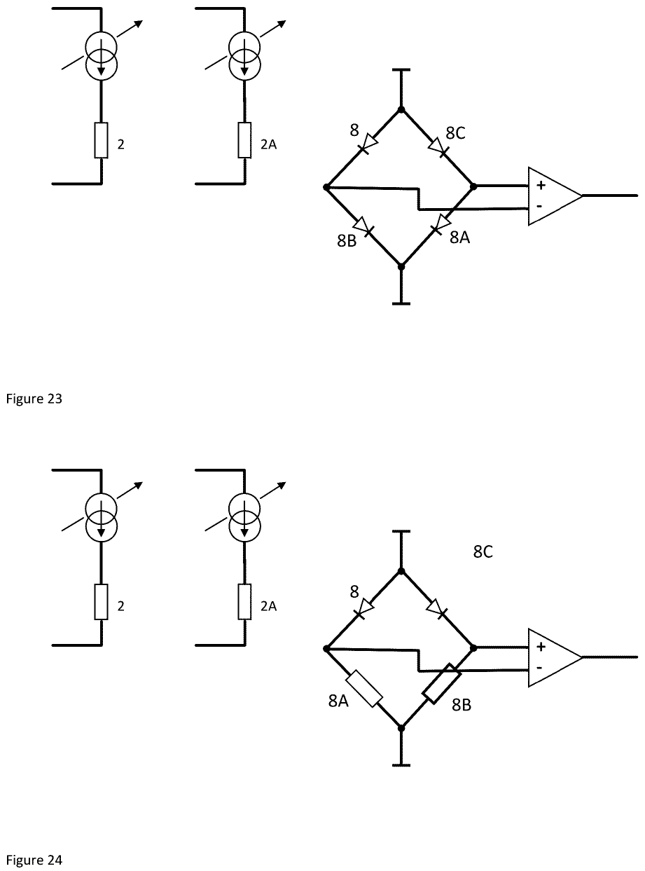

FIG. 23 shows an example circuit for the thermal conductivity sensor shown in FIG. 21. This is similar to the circuit with resistors but the resistors are replaced with diodes.

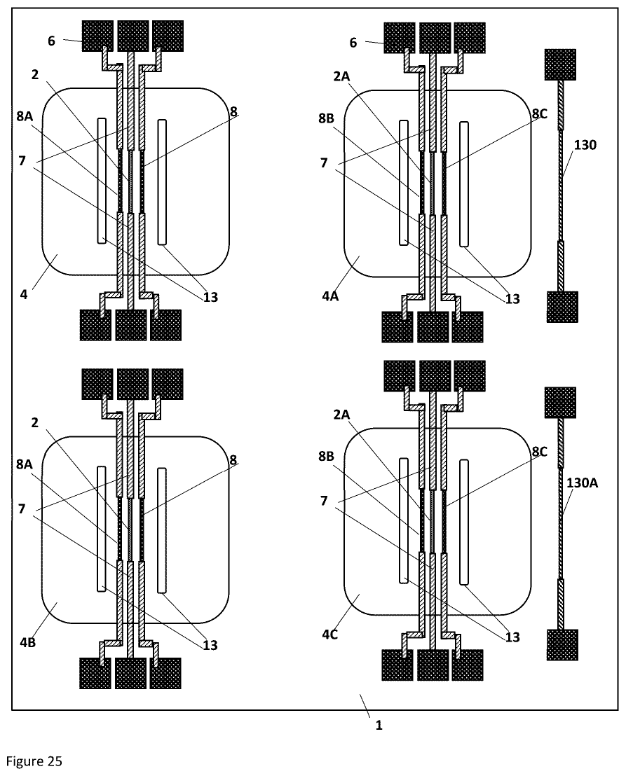

FIG. 24 shows an example circuit for the thermal conductivity sensor shown in FIG. 22. Two of the resistors in the Wheatstone bridge are replaced by diodes. However, the connections of the elements are also changed, where elements 8A and 8B are swapped. This is because of the different behaviour of resistors and diodes. During operation if there is more carbon dioxide present, then the temperature of membrane 4 (and hence temperature sensing elements 8, 8A) will decrease. The resistance of temperature sensing element 8A will decrease, while the forward voltage of the diode 8 will increase. In this case, it's advantageous to have them both in the same branch of the Wheatstone bridge as the effect from both will add together.

FIG. 25 shows an example of a thermal conductivity fluid sensor comprising two pairs of membranes (4, 4A) and (4B, 4C). In this case each pair can be driven at a different temperature and the data can be analysed to improve the selectivity and accuracy of the sensor.

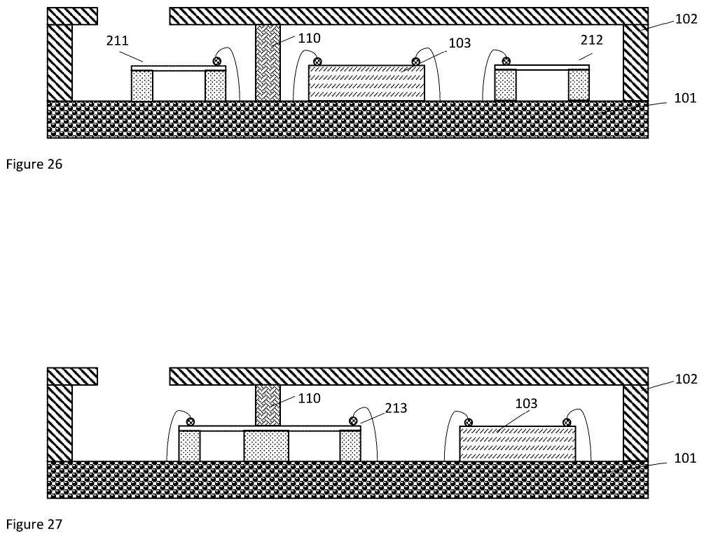

FIG. 26 shows the schematic cross-section of a thermal conductivity sensor package comprising of two sensor chips 111,112 and an ASIC chip 103. The package comprises a base 101, and a lid 102, where the lid has one or more holes. There is a package wall 110 designed such that the package consists of two regions. One region is either hermetically, or semi-hermetically sealed from the ambient environment. The second region has one or more holes in the lid making it open to the environment. The ASIC 103 and one of the sensor chips 112 are placed in the sealed region, and one of the sensor chips 111 is placed in the region open to the environment. Preferably the sensor chips 111 and 112 are identical. In this way sensor chip 112 is always exposed to a known environment, while sensor chip 111 is exposed to the ambient environment, and a differential signal between the two can be used to determine the concentration of the target gas in the ambient environments. The environment in the sealed region can be 100% target gas, a known quantity of target gas in air, synthetic air, pure nitrogen, an inert gas or any other gas or mixture of gases.

FIG. 27 shows the schematic cross-section of a thermal conductivity sensor package comprising a sealed region and an exposed region, but only one sensor chip 113. The wall 110 extend from the lid to the top of chip 113 such that one membrane from chip 113 is in the sealed region, and one membrane is in the exposed region. In this way a single sensor chip can be used while having two regions within the package

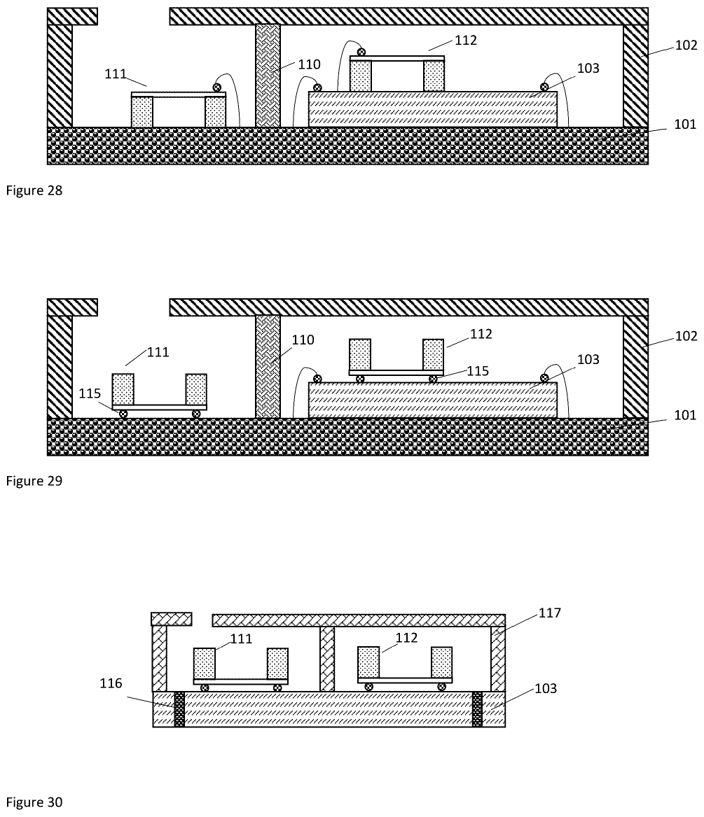

FIG. 28 shows the schematic cross-section of a thermal conductivity sensor package comprising a sealed region and an exposed region, an ASIC chip 103 and two sensor chips 111, 112 where one of the sensor chips 112 is assembled on top of the ASIC chip 103. In this case the foot print of the package is smaller, while the height can be higher.

FIG. 29 show the schematic cross-section of a thermal conductivity sensor package comprising two sensor chips where both the sensor chips 111, 112 are packaged in a flip-chip method. Conductive joints 115 are used to electrically connect the chips to the package or the ASIC. The conductive joints could be solder balls for example. This figure shows the chip 112 above the ASIC, but it could also be side by side with the ASIC and in a flip chip configuration.

FIG. 30 shows another schematic cross-section of a thermal conductivity package with the ASIC chip as the package base. In this case a lid 117 is formed on top of the ASIC chip, and designed such that it separates the package in to a sealed region and an exposed region. There is one sensor chip 112 attached to the ASIC chip by flip chip in the sealed region, and one sensor chip 111 attached to the ASIC chip in the exposed region. Through Silicon Vias (TSVs) 116 within the ASIC chip 103 allow electrical connection to the base of the package. Although this figure show the sensor chips connected by flip chip, it is also possible that the sensor chips are right side up and electrically connected to the ASIC by wire bonds.

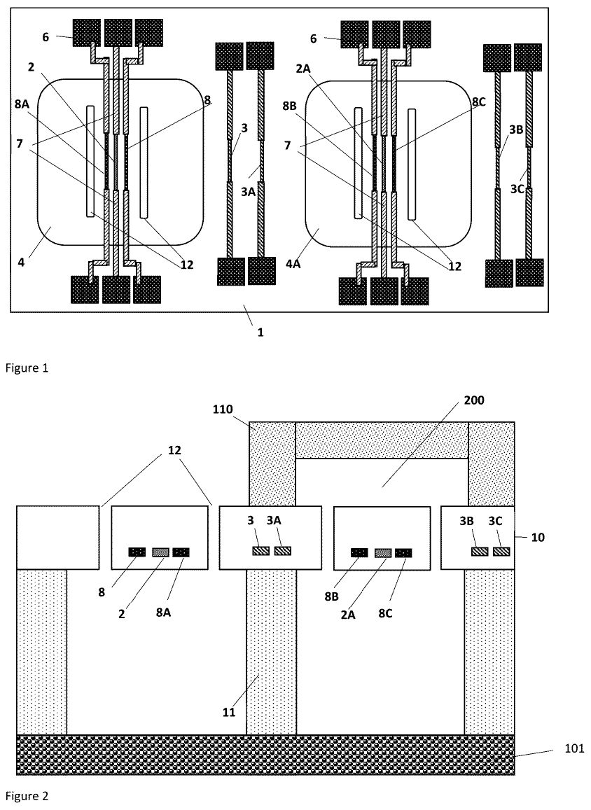

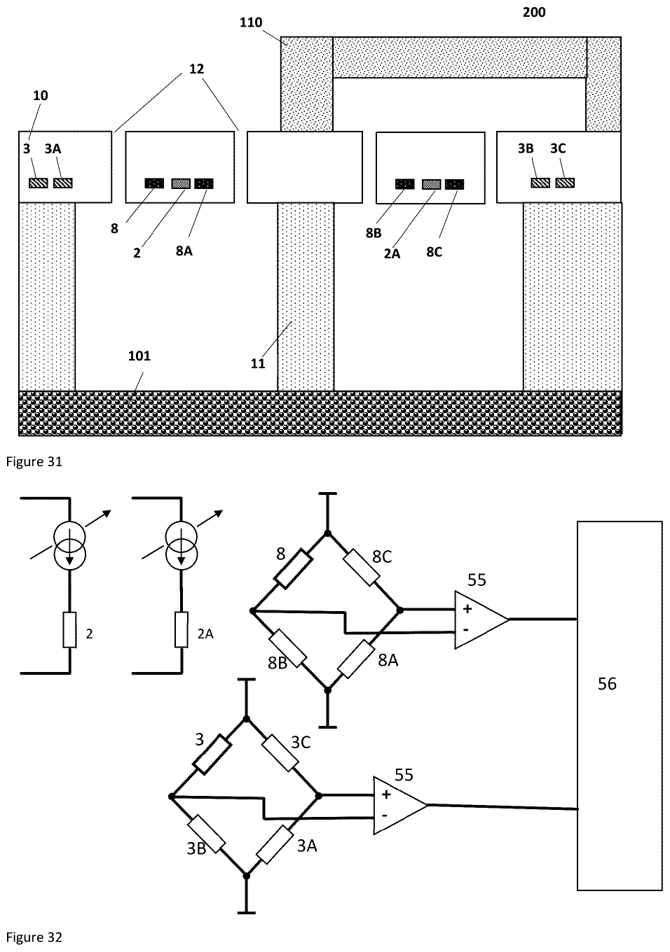

FIG. 31 shows another schematic cross-section of a thermal conductivity sensor. When compared to FIG. 2, the ambient temperature sensors 3, 3A, 3B, 3C are kept distinctly either within the exposed region or in the sealed region. In this example implementation, the temperature sensors 3 and 3A are in the exposed region of the sensor and the temperature sensors 3B and 3C are in the sealed region of the sensor. During operation, the exposed region and the reference region may have a difference in temperature. Although this difference in temperature is likely to be small, it can still be significant enough to affect the measurement results. If the temperatures of the exposed region and the sealed region are known, then this can be used to account for or cancel out the effects of these temperature changes. There are various circuit topologies that can be used to do this, and FIGS. 32-34 illustrate three such example topologies.

In FIG. 32, the ambient temperature sensors 3, 3A, 3B, 3C are part of a Wheatstone bridge, the output of which is connected to a differential amplifier 55. The remaining circuit to drive the heater and the on-membrane temperature sensors may be the same as shown in FIG. 13, and comprise a Wheatstone bridge with temperature sensing elements 8,8A, 8B, 8C, and an instrumentation amplifier 55. The outputs from both of the Wheatstone bridges is provided to a readout circuit block 56 that uses both of the inputs to determine the concentration of the target gas. The circuit block can consist of any kind of circuitry including analog or digital circuitry, as well as memory, microcontroller or microprocessor circuits.

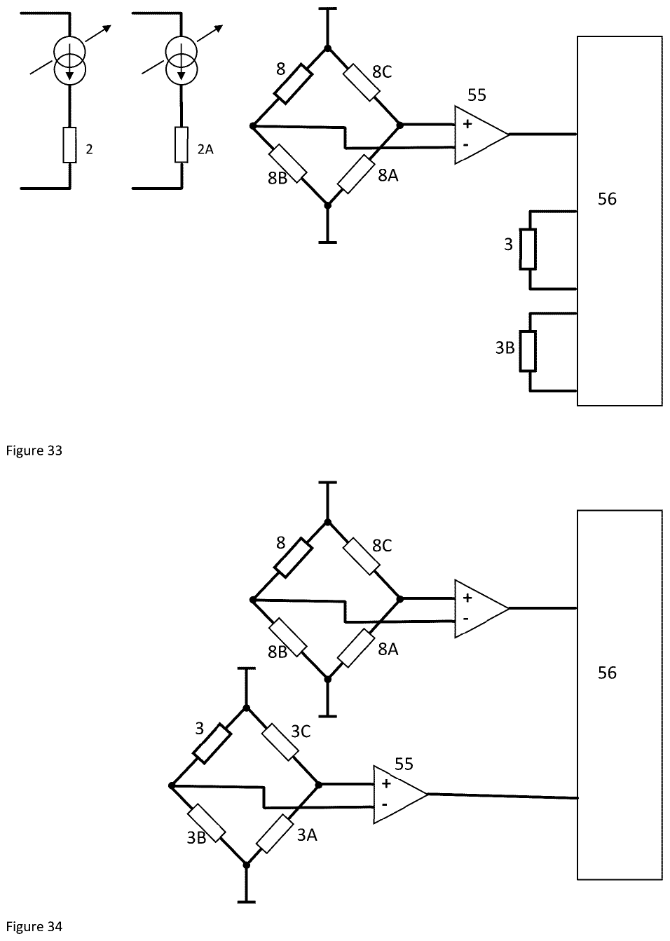

FIG. 33 is an alternate circuit for a device where there is only one temperature sensor 3 in the exposed region, and only one temperature sensor 3B in the sealed region. In this example, the readout circuitry directly reads the temperature from the temperature sensors 3, 3B. The readout circuitry can also include circuitry to measure a differential signal between sensors 3 and 3B.

FIG. 34 is an alternate circuit to FIG. 32, for a device where the on-membrane temperature sensors 8, 8A, 8B, 8C are configured to also act as heaters. The Wheatstone bridge comprising temperature sensors 8, 8A, 8B, 8C both drives the temperature sensors as a heater, and also allows a differential signal between the exposed and sealed sensors to be measured.

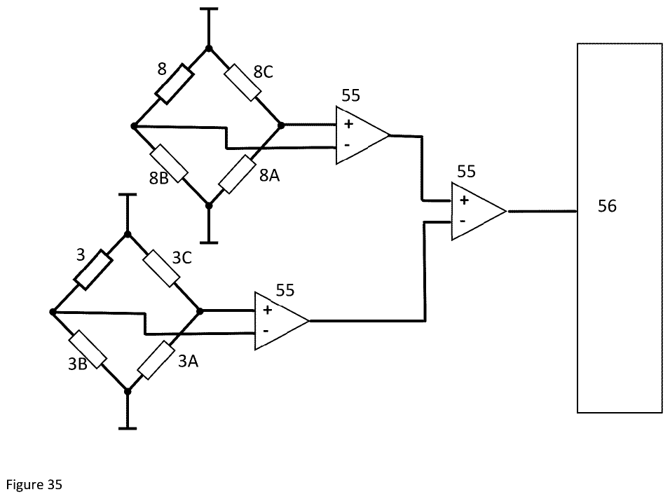

FIG. 35 is a further alternate example circuit compared to FIG. 34, where the differential signal from the on-membrane temperature sensor elements 8, 8A, 8B, 8C, 8D, and the differential signal from the off-membrane temperature sensor elements 3, 3A, 3B, 3C are subtracted from each other using a third differential amplifier 55. This allows the effect of temperature differences between the first and second dielectric membranes to be cancelled out in the circuitry without the need for any further circuitry or signal processing.

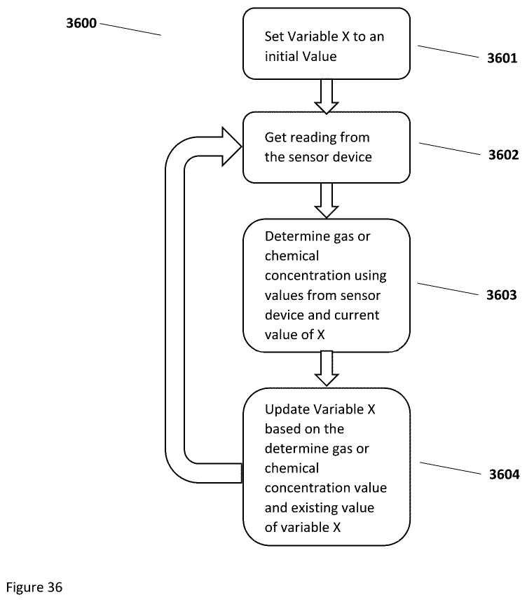

FIG. 36 shows an example flow diagram of a method 3600 to improve the accuracy of a sensor. This method may be used in implementations in which the sealed region is not fully sealed (i.e. in which the region only has a partly or semi-hermetic seal), and may account for how much gas may have leaked into or out of the sealed region. In step 3601 a variable X is set to an initial value. The variable X may represent the amount of target gas in the sealed region. In step 3602, a reading is taken from the sensor device, and this reading along with the variable X is then used to determine the concentration of the target gas or chemical in step 3603. Based on the determined concentration, as well as the current value of variable X, the variable X is then updated in step 3604. Steps 3602-3604 may be repeated by getting another reading from the sensor device, but now using the new value of variable X to determine concentration. It will be understood that the name ‘X’ for the variable is merely provided as an example, and that any variable name is may be used.

It will be understood that the method may be modified in several ways within the scope of the present disclosure. For example, the method may include more than one variable (e.g. ‘X’, ‘Y’, ‘Z’, . . . ) to be updated and used for determining gas concentration rather than just a single variable (‘X’). Additionally or alternatively, the value stored in the one or more variables may be based just on the determined gas concentration without taking into account the existing values of the variable. In implementations using more than one variable, the updated value may be based on the value of one or more of the other variables (either in addition to or instead of the original value for the variable).

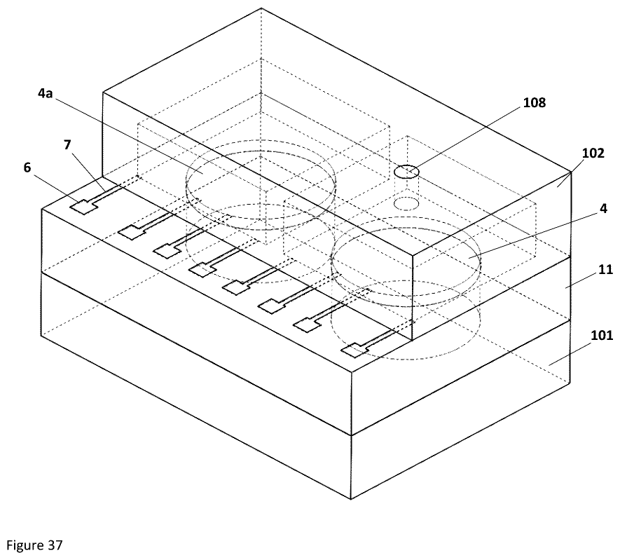

FIG. 37 shows a 3D schematic of the sensor which is formed by wafer bonding. In this example, there is a sensor chip comprising a semiconductor substrate 11 having two etched portions and covered with a dielectric layer. There are two dielectric membranes within the dielectric layer 4, 4a that are adjacent to the etched portions. The membranes have temperature sensors (not shown in diagram for clarity). There is a further substrate 101 provided below substrate 11 to seal the etched portions of substrate 11. A lid 102 is provided above substrate 11. The lid has open volumes above both membranes 4, 4a. However, in case of membrane 4a the open volume is completely sealed, while for membrane 4 there is a hole 108 in the lid 102 that allows air to diffuse in and out from this volume. Bond pads 6 can be used to make electrical connections to other (e.g. external) circuitry, and these are not covered by the lid 102. Tracks 7 provide electrical connections between the bond pads 6 and the temperature sensors on the membranes 4, 4a.

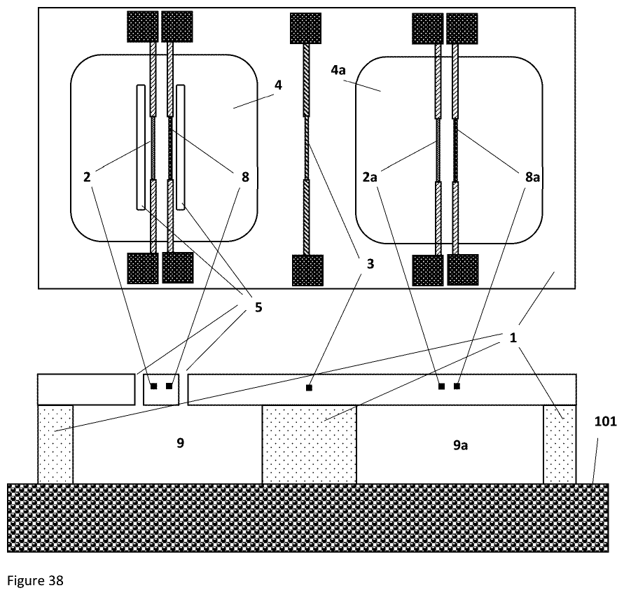

FIG. 38 shows another example thermal conductivity fluid sensor. In this design there are two dielectric membranes, a first membrane 4 and a second membrane 4a. The two membranes are identical, apart from the two (or more) holes 5 on membrane 4. Membrane 4a has no holes. Both membranes have identical heaters 2 and 2a, and sensing elements 8 and 8a. The thermal conductivity fluid sensor is located on a base of a package 3, where the cavities 9 and 9a under the membranes 4 and 4a are both sealed. Such a construction can be designed so that the sensing element 8a, which is exposed to the gas or fluid only on one side of the membrane 4a can be used as a reference for the sensing element 8, which is exposed to the gas on both sides of the membrane 4. This design could lead to a faster and more reliable temperature compensation, since both sensing elements are directly exposed to the same environment, however only sensing element 8 is exposed to the higher gas or fluid concentration.

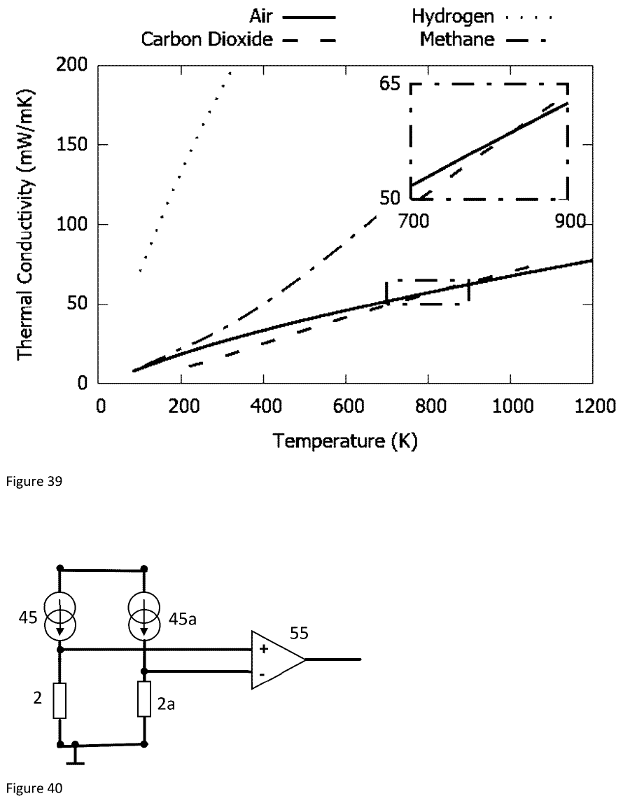

FIG. 39 shows a graph plotting the thermal conductivity of air and different gases across different temperatures. The graph illustrates that if the heater is driven at around 800K, then air and carbon dioxide have the same thermal conductivities, and any deviation from normal is caused by other effects, such as humidity. The heater can then be run at a lower temperature where in addition to other effects, carbon dioxide also causes a deviation in signal. Using algorithms or a look up table the deviation due to other effects can then be cancelled to determine the deviation solely due to carbon dioxide.

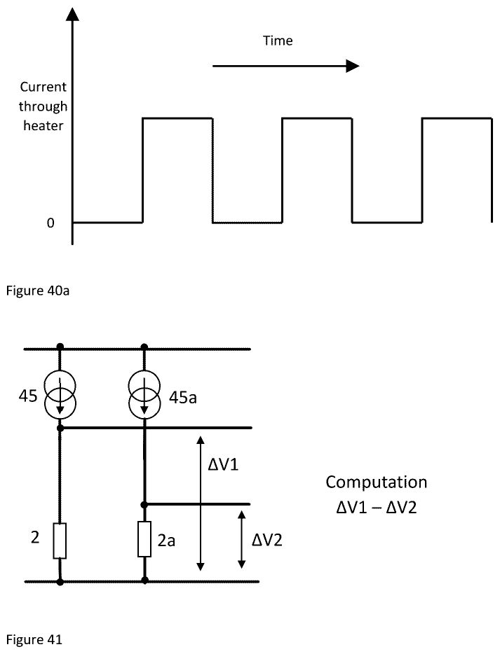

FIG. 40 shows an example circuit for measuring a thermal conductivity fluid sensor such as that shown in FIG. 38. In this circuit two AC current sources 45 and 45a are used with heaters 2 and 2a respectively. Both current sources can independently generate square wave signals, such as the signal shown in FIG. 40a, with an adjustable intensity and frequency. The differential voltage signal, measured across heaters 2 and 2a, is then processed by a lock-in amplifier 55, or a fast Fourier transform (FFT)-based digital signal processing (DSP) circuit.

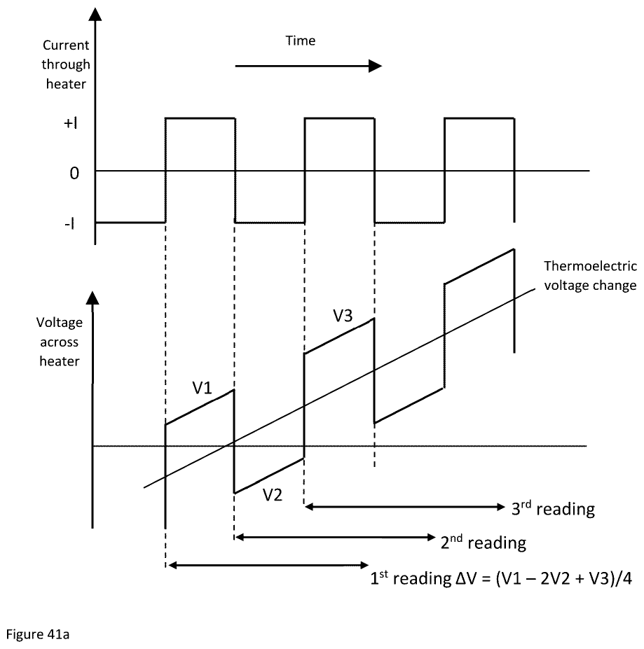

FIG. 41 shows another example circuit for measuring a thermal conductivity fluid sensor such as that shown in FIG. 38. In this circuit two reversible DC current sources 45 and 45a are used with heaters 2 and 2a respectively. Both current sources can independently generate currents with alternating polarities, as shown in FIG. 41a, with adjustable intensity and frequency. In this case the voltage measurements on each heater are performed based on a three-step delta method as detailed in FIG. 41a. This three-step delta method may offer significant advantages over other DC resistance measurement techniques in reducing the impact of or overcoming errors due to changing temperature.

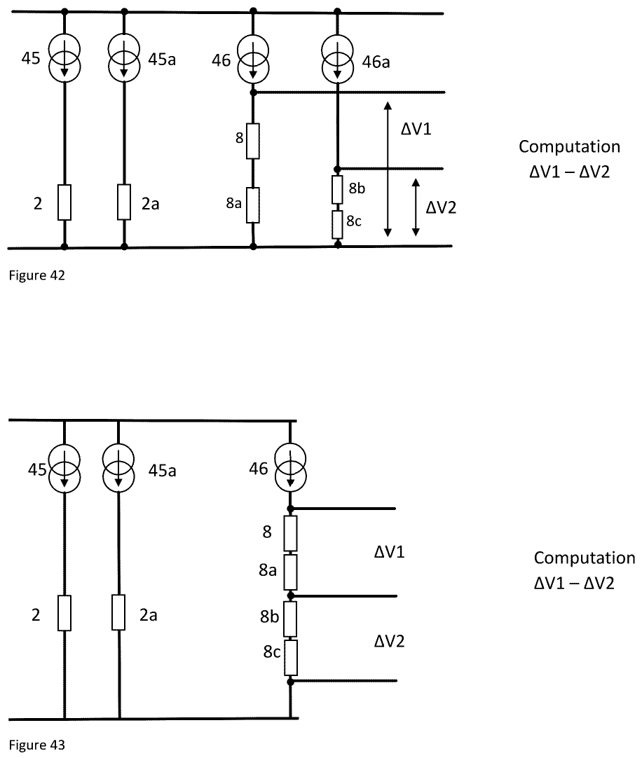

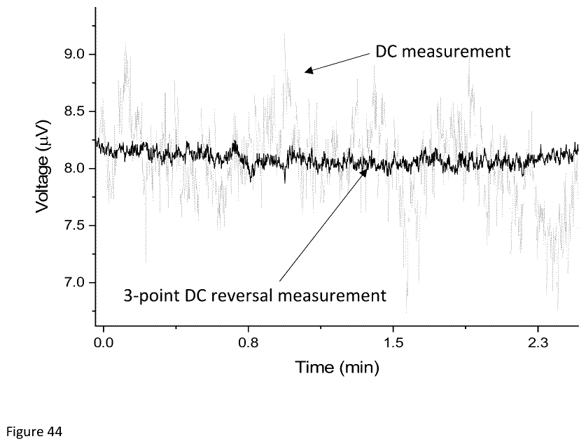

FIG. 42 shows a variation of the circuit presented in FIG. 41. In this circuit two DC current sources 45 and 45a are used with heaters 2 and 2a respectively, while two reversible DC current sources 46 and 46a, providing a much smaller current, are used with sensing elements 8, 8a and 8b, 8c respectively. The much smaller current level provided by the reversible DC current sources 46 and 46a when compared to e.g. the circuit of FIG. 43, aids in reducing transient effects due to current switching and thus may allow for a faster and more accurate delta reading.

FIG. 43 shows variation of the circuit presented in FIG. 42. In this circuit the sensing element 8, 8a and 8b, 8c are connected in series and driven by a single reversible DC current source 46. Using a single current source may improve the circuit immunity to common mode noise while simplifying circuit overall.

FIG. 44 shows a comparison between ˜1200 DC voltage measurements of a ˜60Ω heater made with ˜8 mA test current taken approximately over 120 seconds. The DC measurements fluctuate with a voltage error of up to 30%, whereas the three-point DC reversal method measurements fluctuate with less than 5% error. These figures can be further significantly improved by using a smaller (e.g. less than a few μA) test current.

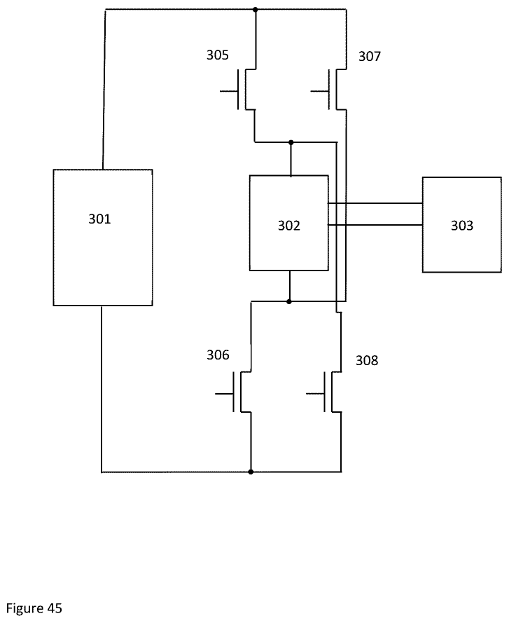

FIG. 45 shows a circuit schematic for driving the sensors in two different current directions. There is a control 301 that provides the electrical bias to the sensors 302. The sensors are read by a read out circuit 303. The transistors 305, 306, 307 and 308 control the direction of current within the sensors 302. When transistors 305 and 306 are on, and 307 and 308 are off then the current flows in one direction through the sensors. While when the transistors 305 and 306 are off, and transistors 307 and 308 are on, the current flows in the opposite direction. This method can be used to improve the accuracy of measurements using the delta method. The control system 301 can be just a current source, or a voltage source, or a more complex circuit. The sensors 302 may be e.g. a temperature sensing resistor, or may comprise more than one resistor. For example, the more than one resistors may be provided in a bridge configuration—where either all the branches have active sensors, and/or some branches have fixed resistors. The read out circuit 303 can have a differential amplifier, filter and/or an analog to digital circuit.

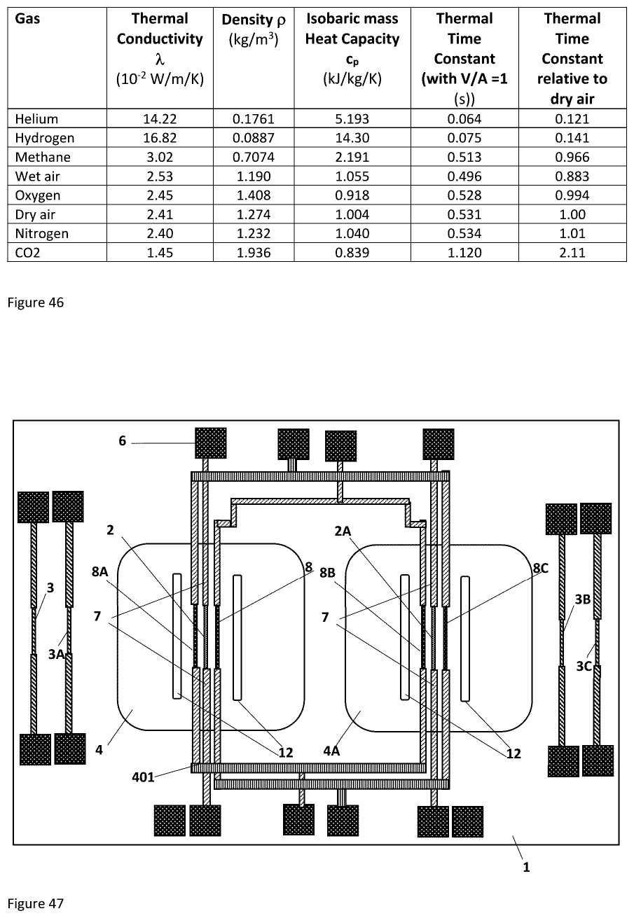

FIG. 46 provides the thermal properties of different example gases of interest as well as those of dry and wet air (at standard temperature and pressure). The values of nitrogen and oxygen are also provided to demonstrate how sensitive the values can be to the oxygen content of air.

FIG. 46 also shows the thermal response time relative to dry air for each of these gases. For example, it can now be seen that Helium gas is 8.3× faster than dry air and 7.1× faster than wet air. It can be seen that hydrogen gas is 6.9× faster than methane. Wet air is 1.13 or 13% faster than dry air. Finally, it can be seen that CO2 is 2.1× slower than dry air and 2.4× slower than wet air.

It is now possible to determine the gas type from the thermal response time and hence, knowing the gas, also determine the concentration of the gas in air. It is also possible to determine the different gases in the mixture because there will be two distinct thermal constants in the TC response. For example, one faster one for H2 and one much slower one (×6.9) for CH4.

Driving the thermal conductivity heater with an AC signal (or using a pulse) will result in different frequency responses according to the gas type and concentration. The frequency content of the signal (e.g. an FFT) will show which gas is present by a characteristic frequency and the height of the FFT peak will give its concentration. In this way we can determine the type of gas when the gas is unknown and also determine the gases present in a gas mixture as well as their concentrations.

Finally, it should be noted that the thermal time constant of dry and wet air are similar (12% difference) and very different to that of H2 and CO2. In other words, the relative humidity of the air will not significantly affect the signal at the frequency for CO2 or H2.

This AC method decouples the thermal signal of the target gas (for example CO2) from that of a variable background gas (for example, other components of air such as nitrogen and/or oxygen), and therefore provides a much more accurate way of measuring a gas concentration or composition than DC techniques.

FIG. 47 shows a top view of a thermal conductivity fluid sensor with two membranes and two temperature sensors on each membrane. The temperature sensors 8, 8A, 8B, 8C are connected together as a bridge (such as a Wheatstone bridge) on the sensor chip. An additional metal layer 401 may be used to help with the routing of the connections. The additional metal layer 401 and any other connections between the temperature sensors may be formed on or within the dielectric layer or semiconductor substrate.

Such a configuration on the sensor chip can be used in conjunction with a separate ASIC chip. Alternatively, it can be used along with discrete (component based) circuitry. Forming the Wheatstone bridge connections between the temperature sensing elements on the sensor chip can result in better symmetry within the bridge. Additionally, parasitics and noise can be minimised. Furthermore, reliability may be improved and complexity reduced as a lower component count and less external connections are needed. This example shows separate heating elements and temperature sensing elements. However it will be understood that the heating elements may be omitted and the temperature sensing elements may be used as heating elements while they are connected up in a bridge configuration.

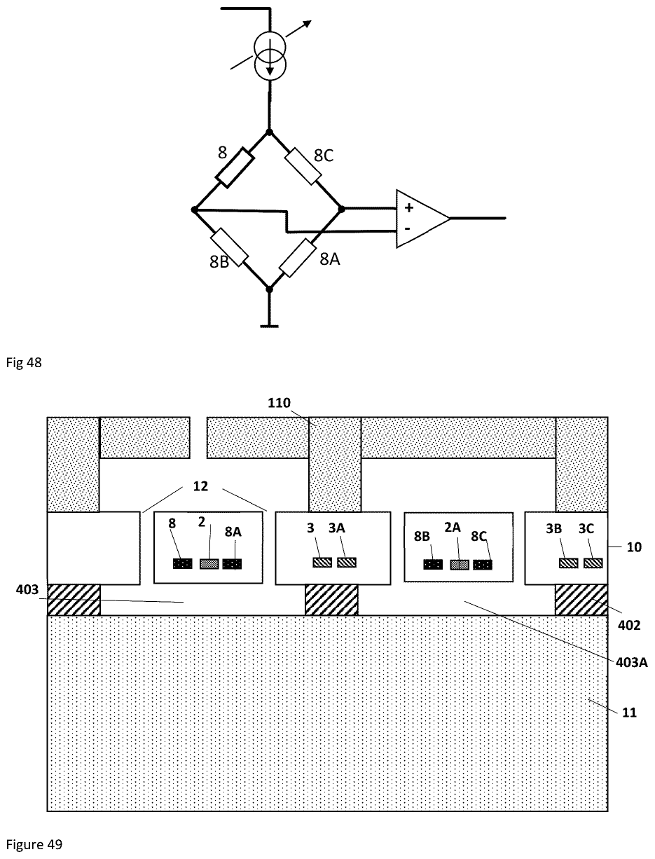

FIG. 48 shows a circuit diagram of a Wheatstone bridge driven in constant current mode. In this circuit, the temperature sensing elements are used as heating elements, and the heating elements are connected in a Wheatstone bridge configuration. The bridge is driven through a constant current supply, and a differential measurement is taken on the Wheatstone bridge.

FIG. 49 shows a cross-section schematic of a thermal conductivity fluid sensor with an additional layer between the dielectric layer and the substrate, provided with a cavity portion. In this example, there is an additional layer 402 located between the dielectric layer and the substrate. In this case, there is no cavity within the substrate, but there are cavities 403 and 403A within the additional layer 402, and the dielectric membranes are over the cavities 403 and 403A formed within the additional layer. Such a structure can be formed by bulk micromachining and allows very controlled and thin gaps to be provided below the membrane, resulting in improved sensitivity to thermal conductivity of the target gas.

Layer 402 may comprise more than one layers, and one or more of the layers may have a cavity within them. Furthermore, in addition to the cavities in layer 402, there may be a further cavity in the substrate, e.g. for increased resistance to manufacturing tolerances.

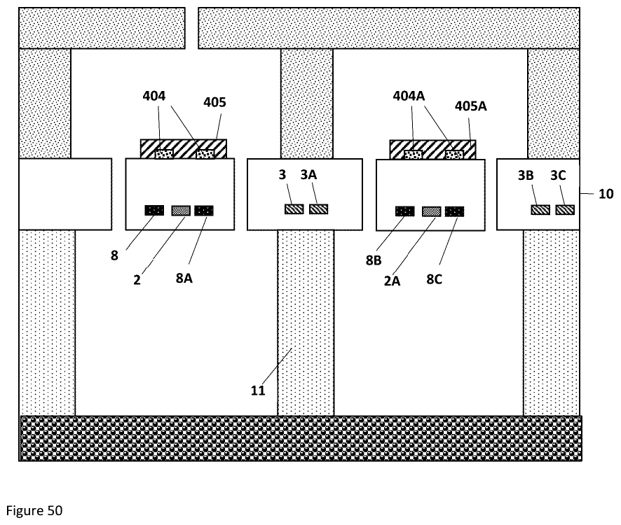

FIG. 50 shows schematically a cross-section of a further thermal conductivity fluid sensor having a fluid sensitive material and electrodes in contact with the fluid sensitive material. The thermal conductivity sensor includes electrodes 404 and 404A, and fluid sensing layers 405 and 405A comprising a fluid sensitive material. The electrodes 404, 404A may be made of aluminium, platinum, gold, tungsten or any other metal. The electrodes 404, 404A may be used as a humidity sensor, the sensing materials 405 and 405A may comprise alumina or a different humidity sensitive material. The presence of humidity may change the dielectric constant of layers 405/405A, and hence the capacitance on the electrodes 404, 404A which can be measured to determine the humidity.

Humidity in the sealed region is constant as long as the atmosphere in the sealed region is not altered. The reading of the humidity sensor located in the sealed region may be used as a reference for the humidity sensor located in the exposed region. The advantage of having humidity sensors is that they allow to cancel any effect of humidity on the fluid sensing measurements.

Alternately, sensing layers 405 and 405A may comprise a material which is sensitive to other gases. In this case, the impedance (resistance and/or capacitance) of the sensing layers 405 and 405A, may change in the presence of a certain gas at a certain temperature, because of a chemical reaction between the gas and the sensing material and/or a diffusion of the gas into the sensing layer.

Electrodes 405, 405A may be further used to detect a leakage in the sealed reference chamber, during operation and lifetime of the product, caused by a defect in the seal. Any such leak may affect the accuracy of the sensor reading when the sensor reading is a result of the differential measurement between an active sensing element exposed to a fluid and a reference sensor located in the sealed chamber.

For example, if the reference sensing element is sealed in nitrogen, a leak in the seal may cause oxygen from the external atmosphere to enter the sealed chamber so changing the composition of the atmosphere in the reference chamber and, in turn, a signal produced by the reference sensing element. If the sensing layers 405 and 405A are sensitive to oxygen, then the leak can be detected, and appropriate actions may be taken, for example applying suitable compensation so that an accurate sensor signal may be produced.

The sensing layers 405 and 405A may comprise a metal oxide, such as tungsten oxide, which is sensitive to oxygen. The sensing layers 405, 405A may be made of any other suitable material known in the art.

Alternatively, the sensing layers 405 and 405A may comprise a material which is sensitive to a gas which forms the atmosphere of the reference chamber. In this case if, due to a leak, the gas escapes the reference chamber, its concentration within the chamber drops and this change in concentration may be detected by the sensing layers 405 and 405A. Such configuration allows self-calibration of the device by monitoring the changes in the reference atmosphere.

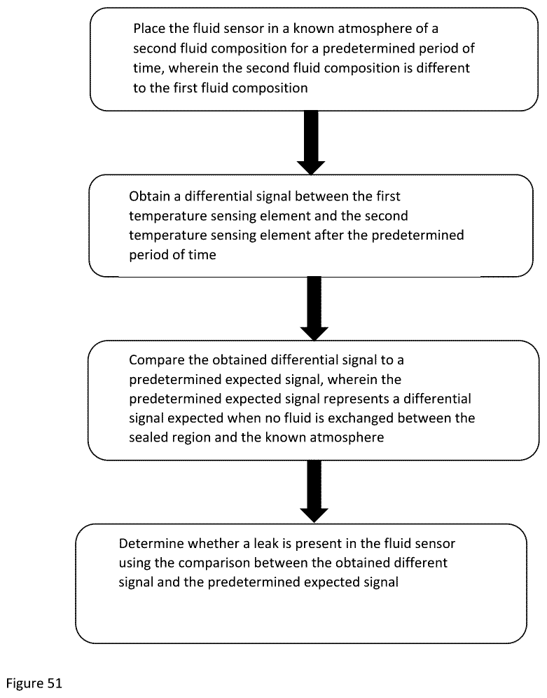

FIG. 51 shows a flow chart of a method for testing a thermal conductivity fluid sensor as described herein. The method may be used to test a thermal conductivity sensor with a sealed reference chamber comprising a reference atmosphere of a first fluid composition, for leaks in the seal of the reference chamber.

The method includes steps of:
i. placing the fluid sensor in a known atmosphere of a second fluid composition for a predetermined time period, wherein the second fluid composition is different to the first fluid composition. This exposes the sensor to the known atmosphere that is different, in composition and/or pressure from the atmosphere of the reference chamber. The first fluid composition and/or the second fluid composition may each include a vacuum, a gas, or a liquid. For example, the reference chamber may be under a vacuum, or may be at any suitable pressure and may comprise dry air, nitrogen, hydrogen, carbon dioxide, argon and/or any other suitable gas. Similarly, the known atmosphere of the second fluid composition may be a vacuum, or it may comprise dry air, nitrogen, hydrogen, carbon dioxide, argon and/or any other gas, at any suitable pressure;
   in this way, if a leak is present in the seal of the reference chamber, at least some fluid may be exchanged between the reference chamber and known atmosphere, causing a change in the reference atmosphere;
ii. obtaining a differential signal between the first temperature sensing element and the second temperature sensing element after the predetermined period of time;
iii. comparing the obtained differential signal to a predetermined expected signal, wherein the predetermined expected signal represents a differential signal expected when no fluid is exchanged between the sealed region and the known atmosphere. The differential signal may be a reference signal;
iv. determining whether a leak is present in the fluid sensor using the comparison between the obtained different signal and the predetermined expected signal.

By taking a differential sensor measurement and comparing it to the value expected in the presence of the known atmosphere if no leaks were present, it is possible to determine whether the reference chamber and, in turn, the device is leaking. If the expected signal and the obtained signal are the same, no leak are present but if they are different, a leak is present.

The method may include taking one or more differential readings after a predetermined time and comparing them to an expected value. Alternatively, the method may include taking a series of readings and comparing them to previous readings, to detect if there is a progressive change of signal due to a progressive change in the reference atmosphere with time. Taking a series of readings can be used to detect smaller leaks.

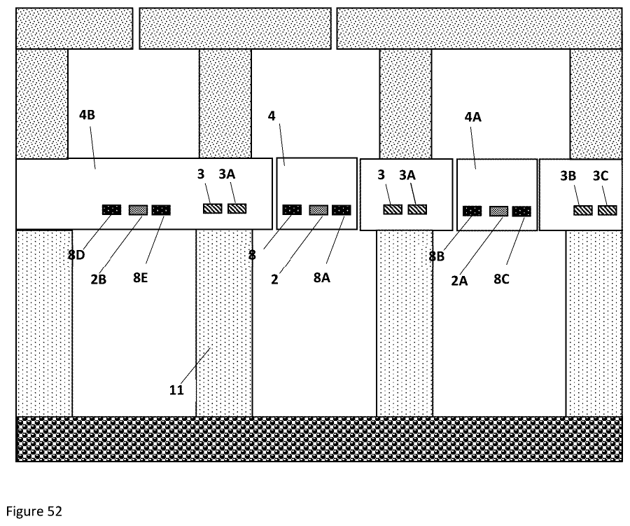

FIG. 52 shows schematically a cross-section of a thermal conductivity fluid sensor having three dielectric membranes. In use, a first reference membrane is sealed from the atmosphere, a second membrane has holes and is exposed to the atmosphere, and a third reference membrane is exposed to the atmosphere from a top surface of the membrane, but is sealed on a lower surface of the membrane.

A first membrane 4A has holes and is sealed from the atmosphere and acts as a reference membrane. A second membrane 4 has holes and is exposed to the atmosphere from above and below the membrane. A third membrane 4B is exposed to the atmosphere only on the upper side or surface of the dielectric membrane, as it has no holes. This means that the third membrane 4B is sealed from the atmosphere on a lower surface of the membrane. A differential measurement between the temperature sensing elements of membranes 4 and 4A may respond differently to the atmosphere compared to a differential signal between the temperature sensing elements of membranes 4 and 4B, for example there may be a stronger humidity coupling between the elements of membrane 4B and 4 compared to 4A and 4. This means that different information may be obtained when membrane 4 has as reference the membrane 4A compared to when the reference membrane is 4B.

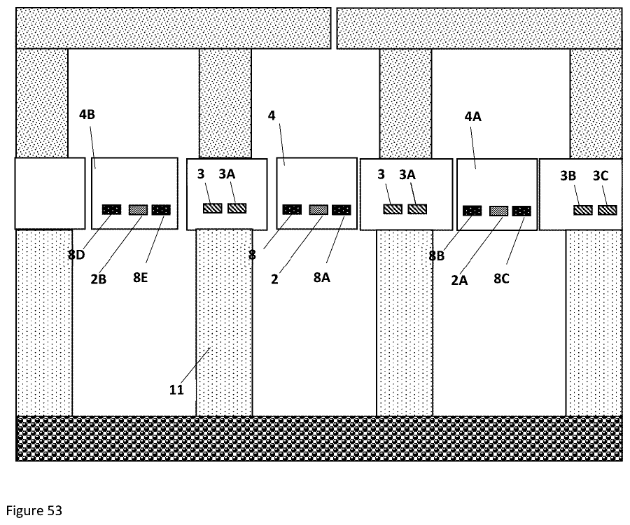

FIG. 53 shows schematically a cross-section of a thermal conductivity fluid sensor having three dielectric membranes. In use, first and second reference membranes 4A and 4B are sealed from the atmosphere, and a sensing membrane 4 is exposed to the atmosphere. The further sealed reference membrane 4B, which can be sealed with a vacuum atmosphere may be provided to be used as a lifetime monitoring structure during the operation of the sensor.

The three dielectric membranes are enclosed in three corresponding chambers. Membrane 4 has holes and is exposed to the atmosphere from above and below. Membrane 4A has holes and is sealed from the atmosphere and acts as a reference membrane, e.g. for differential measurements. Membrane 4A is enclosed in a sealed reference chamber. Membrane 4B is similarly sealed from the atmosphere and acts as an additional reference membrane. Membrane 4B is enclosed in an additional reference chamber. If membrane 4A is sealed in a known atmosphere, such as dry air, and membrane 4B is sealed in vacuum (or in a different known atmosphere to the one of membrane 4A), then this configuration may be used to (indirectly) check for leaks in the seal of the reference chamber 4A during lifetime operation.

During the sensor operation, leaks in the seal of the reference chamber 4B may be detected by monitoring (e.g. periodically) the vacuum level within the vacuum sealed further reference chamber enclosing membrane 4B. This could be done by measuring the temperature of the heating element 2B provided to membrane 4B (or the temperature of a temperature sensing element placed in the vicinity of such heating element 2B) for a given power level applied to the heating element. In the presence of a leak, the temperature of the heating element would gradually decrease (compared to the value expected when no leak is present). This may provide an indication that the seal of the reference chamber, enclosing membrane 4A (sealed in dry air) may have failed too, since they have both been used for the same time period in similar conditions, and, therefore, the heating element provided to membrane 4A may no longer provide a solid reference to the active heating element provided to membrane 4, for a differential or bridge measurement.

It will be understood that, while the further reference membrane 4B may preferably be sealed in a vacuum, in other embodiments it may be sealed in any other suitable atmosphere that is different from the application atmosphere, to which membrane 4 is exposed, or air. For example, it may be sealed in low pressure air, high pressure air, hydrogen, helium, argon and/or carbon dioxide or any other suitable gas or combination of gases at any suitable pressure. Different pressures or gases may be used as long as they behave in a significantly different way from air, or the application atmosphere, with respect of their interaction with the heater.

The skilled person will understand that in the preceding description and appended claims, positional terms such as ‘above’, ‘overlap’, ‘under’, ‘lateral’, etc. are made with reference to conceptual illustrations of a device, such as those showing standard cross-sectional perspectives and those shown in the appended drawings. These terms are used for ease of reference but are not intended to be of limiting nature. These terms are therefore to be understood as referring to a device when in an orientation as shown in the accompanying drawings.

Although the disclosure has been described in terms of preferred embodiments as set forth above, it should be understood that these embodiments are illustrative only and that the claims are not limited to those embodiments. Those skilled in the art will be able to make modifications and alternatives in view of the disclosure which are contemplated as falling within the scope of the appended claims. Each feature disclosed or illustrated in the present specification may be incorporated in the disclosure, whether alone or in any appropriate combination with any other feature disclosed or illustrated herein.

Reference Numerals

| | |
|---|---|
| 1 Semiconductor chip | 7 Tracks |
| 2 Heating element | 8, 8A, 8B, 8C, 8D, 8E temperature |
| 2A Heating element | sensing element |
| 3, 3A, 3B, 3C temperature sensing | 9 Etched portion under membrane |
| element outside the membranes | 9a Sealed etched portion under |
| 4 Dielectric membrane | membrane |
| 4A Additional dielectric membrane | 10 Dielectric layer |
| 4A Further additional dielectric | 11 Semiconductor substrate |
| membrane | 12 Membrane Recess |
| 6 Bond pads | 14 Second dielectric membrane |
| 20 Connecting element | 106 Inlet |
| 25 Covering layer | 107 Outlet |
| 26 Fluid channel above membrane | 108 Hole through package lid |
| 30 Hole through covering layer | 110 Lid |
| 35 Printed Circuit Board | 111 Instrumentation amplifier |
| 36 Solder balls | 115 Solder Balls |
| 40, 41 Additional resistor | 116 Through Silicon Vias |
| 42, 43 Additional resistor | 117 Lid for a chip scale package |
| 44 Variable resistor | 130 Ambient Temperature sensor |
| 45, 45a, 46, 46a, 47 Current source | 140, Sealant for flip chip package |
| 50 Reference voltage | 200 Sealed cavity |
| 55 Differential amplifier | 210A Differential Amplifier |
| 56 Read out Circuitry | 210B Differential Amplifier |
| 60 Ground | 211 Sensor die in Exposed Region |
| 65 Field Effect Transistor | 212 Sensor die in sealed region |
| 70 Switch | 213 Combined sensor chip in package |
| 75 Ambient temperature sensing | 301 Control Circuitry |
| element | 302 Sensor(s) |
| 80 Heater control | 303 Read out Circuitry |
| 100 Pair of temperature sensing | 305, 306, 307, 308 switching transistors |
| elements | 401 Additional metal layer |
| 101 Package base | 402 Additional layer |
| 102 Package lid | 403 Cavities |
| 103 ASIC | 404 Electrodes |
| 104, 105 Wire bonds | 405 Fluid sensitive layer |

1. A fluid sensor for sensing a concentration or composition of a fluid, the sensor comprising:
a semiconductor substrate comprising a first substrate portion and a second substrate portion, the first substrate portion comprising a first cavity and the second substrate portion comprising a second cavity;
a dielectric region located on the semiconductor substrate, wherein the dielectric region comprises a first dielectric membrane provided on the first cavity and a second dielectric membrane provided on the second cavity;
a first temperature sensing element located on or within the first dielectric membrane and a second temperature sensing element located on or within the second dielectric membrane;
an output circuit configured to measure a differential signal between the first temperature sensing element and the second temperature sensing element,
wherein, the fluid sensor comprises a first region configured to be exposed to the fluid, and a second region configured to be isolated from the fluid,
wherein the first dielectric membrane is located in the first region, such that in use, the first dielectric membrane is exposed to the fluid, and
wherein the second dielectric membrane is located in the second region such that in use, the second dielectric membrane is isolated from the fluid.

2. A fluid sensor according to claim 1, wherein the fluid sensor comprises:
a first set of temperature sensing elements each located on the first membrane, wherein the first set of temperature sensing elements comprises the first temperature sensing element and a third temperature sensing element; and
a second set of temperature sensing elements each located on the second membrane, wherein the second set of temperature sensing elements comprises the second temperature sensing element and a fourth temperature sensing element, and
wherein the output circuit is configured to measure a differential signal between the first set of temperature sensing elements and the second set of temperature sensing elements.

3. A fluid sensor according to claim 2, wherein the output circuit comprises a Wheatstone bridge, and wherein the first set of temperature sensing elements operate as the first and second legs of the Wheatstone bridge and wherein the second set of temperature sensing elements operate as the third and fourth legs of the Wheatstone bridge.

4. A fluid sensor according to claim 3, wherein the Wheatstone bridge is formed on a same chip as the fluid sensor; and
optionally further comprising electrical connections between the first set of temperature sensing elements and the second set of temperature sensing elements, and wherein said electrical connections are on or over the dielectric region.

5. A fluid sensor according to claim 1, further comprising:
a first fluid sensitive layer located on or over the first dielectric membrane;
a second fluid sensitive layer located on or over the second dielectric membrane;
a first electrode in contact with the first fluid sensitive layer; and
a second electrode in contact with the second fluid sensitive layer.

6. A fluid sensor according to claim 5, further comprising a control unit configured to receive a resistive or capacitive reading from the first electrode and/or the second electrode, and wherein the control unit is configured to calibrate the sensor using the received resistive or capacitive reading.

7. A fluid sensor according to claim 5, wherein the fluid sensor comprises:
a first set of electrodes in contact with the first fluid sensitive layer, wherein the first set of electrodes comprises the first electrode and a third electrode separated from each other by at least a portion of the first fluid sensitive layer; and
a second set of electrodes in contact with the second fluid sensitive layer, wherein the second set of electrodes comprises the second electrode and a fourth electrode separated from each other by at least a portion of the second fluid sensitive layer; and
optionally wherein a resistance or capacitance of the first fluid sensitive layer and the second fluid sensitive layer is dependent on humidity.

8. A fluid sensor according to claim 1, wherein the semiconductor substrate comprises separate first and second semiconductor substrate sections; or
wherein the semiconductor substrate comprises integral first and second substrate sections,
and wherein the first dielectric membrane and the second dielectric membrane comprise integral sections of a dielectric layer.

9. A fluid sensor according to claim 1, wherein the first temperature sensing element and the second temperature sensing element are configured to operate as heating elements.

10. A fluid sensor according to claim 9, comprising a controller configured to:
drive one or more of the heating elements in an AC mode to modulate the temperature of the heating elements to vary the differential signal;
monitor the differential signal at the modulation frequency using a lock-in amplifier and/or based on a Fourier transform-based technique; and
selectively differentiate between different fluid components and/or determine the concentration of the different fluid components based on the differential signal.

11. A fluid sensor according to claim 9, comprising two DC current sources configured to independently generate DC currents with alternating polarities; and a controller configured to:
drive one or more of the heating elements or a sensing element adjacent to the heating element in using DC pulses from the two DC current sources; and
monitor the differential signal based on a two point or a three point DC reversal-based technique; and
selectively differentiate between different fluid components and/or determine the concentration of the different fluid components based on a differential signal; or
comprising a control unit configured to drive one or more of the heating elements in an AC bias or a pulse bias, and further configured to determine the concentration and type of gas or gases present based on the frequency content of a resulting signal.

12. A fluid sensor according to claim 9, comprising one or more current sources with alternating polarities, and a controller configured to:
drive one or more of the heating elements using a current from the one or more current sources; and
monitor the differential signal based on a two point or a three point DC reversal-based technique; and
selectively differentiate between different fluid components and/or determine the concentration of the different fluid components based on a differential signal; and
optionally wherein one or more of the heating elements is driven by a single current source, and wherein the fluid sensor comprises switches configured to change the direction of a current in terminals of the heating element or the sensing element.

13. A fluid sensor according to claim 1, further comprising a first ambient temperature sensor close or adjacent to the first dielectric membrane and a second ambient temperature sensor close or adjacent to the second dielectric membrane, and
wherein the fluid sensor is configured to determine a measurement of the fluid concentration or composition based on at least one of:
(i) a signal from the first ambient temperature sensor;
(ii) a signal from the second ambient temperature sensor; and/or
(iii) a differential signal between the first ambient temperature sensor and the second ambient temperature sensor.

14. A fluid sensor according to claim 1, further comprising a first ambient temperature sensor and a second ambient temperature sensor,
wherein the first ambient temperature sensor is located in the first region, and wherein the second ambient temperature sensor is located within the second region; and
wherein the fluid sensor is configured to determine a measurement of the fluid concentration or composition based on at least one of:
(i) a signal from the first ambient temperature sensor;
(ii) a signal from the second ambient temperature sensor; and/or
(iii) a differential signal between the first ambient temperature sensor and the second ambient temperature sensor.

15. A fluid sensor according to claim 1, wherein the fluid sensor comprises a measurement system configured to:
store one or more variables;
receive a reading from one or more of the temperature sensing elements;
determine the fluid composition based on the reading and the one or more variables; and
update the one or more variables based on the reading and/or the determined fluid composition.

16. A fluid sensor according to claim 1, wherein the second region comprises a sealed region comprising a fluid of a known composition.

17. A fluid sensor according to claim 1, wherein the semiconductor substrate further comprises a third substrate portion comprising third cavity, and
wherein the dielectric region comprises a third dielectric region provided on the third cavity, and
wherein the fluid sensor comprises a third temperature sensing element located on or within the third dielectric membrane; and
optionally wherein the third dielectric membrane is located in a third region configured to be isolated from the fluid, and wherein the third region comprises a sealed region comprising a vacuum; and
optionally comprising an output circuit configured to measure a signal from the third temperature sensing element, and wherein the fluid sensor is configured to calibrate the differential signal using the measured signal from the third temperature sensing element; or
wherein a first surface of the third dielectric membrane is exposed to the fluid and wherein a second surface of the dielectric membrane is isolated from the fluid.

18. A fluid sensor for sensing a concentration or composition of a fluid, the sensor comprising:
a semiconductor substrate comprising a first cavity and a second cavity;
a dielectric region located on the semiconductor substrate, wherein the dielectric region comprises a first dielectric membrane located over the first cavity of the semiconductor substrate, and a second dielectric membrane located over the second cavity of the semiconductor substrate;
two temperature sensing elements on or within the first dielectric membrane and two temperature sensing elements on or within the second dielectric membrane;
an output circuit configured to measure a differential signal between the two temperature sensing elements of the first dielectric membrane and the two temperature sensing elements of the second dielectric membrane;
wherein the first dielectric membrane forms a first sealed region that, in use, is exposed to the fluid via one or more holes; and
wherein the second dielectric membrane forms a second sealed region, wherein the second sealed region is isolated from the fluid when in use.

19. A method of testing a fluid sensor according to claim 1, wherein the second region comprises a sealed region and wherein the sealed region comprises a first fluid composition, the method comprising:
placing the fluid sensor in a known atmosphere of a second fluid composition for a predetermined period of time, wherein the second fluid composition is different to the first fluid composition;
obtaining a differential signal between the first temperature sensing element and the second temperature sensing element after the predetermined period of time;
comparing the obtained differential signal to a predetermined expected signal, wherein the predetermined expected signal represents a differential signal expected when no fluid is exchanged between the sealed region and the known atmosphere;
determining whether a leak is present in the fluid sensor using the comparison between the obtained different signal and the predetermined expected signal.

20. A method of manufacturing a fluid sensor for sensing a concentration or composition of a fluid, the method comprising:
forming a semiconductor substrate comprising a first substrate portion and a second substrate portion, the first substrate portion comprising a first etched portion and the second substrate portion comprising a second etched portion;
forming a dielectric region located on the semiconductor substrate, wherein the dielectric region comprises a first dielectric membrane provided on the first etched portion and a second dielectric membrane provided on the second etched portion;
forming a first temperature sensing element located on or within the first dielectric membrane and a second temperature sensing element located on or within the second dielectric membrane;
forming an output circuit configured to measure a differential signal between the first temperature sensing element and the second temperature sensing element,
wherein, the fluid sensor comprises a first region configured to be exposed to the fluid, and a second region configured to be isolated from the fluid,
wherein the first dielectric membrane is located in the first region, such that in use, the first dielectric membrane is exposed to the fluid, and
wherein the second dielectric membrane is located in the second region such that in use, the second dielectric membrane is isolated from the fluid.
